SEQUENCE LISTING (2 sequences)

SEQ ID NO: 1 - DNA, 1982 bp, Homo sapiens
gcagaggaggagcgcacgccggcctcgaagaacttctgcttgggtggctgaactctgatcttgacctagagtcatggccatggcaaccaaaggaggtactgtcaaagctgcttcaggattcaatgccatggaagatgcccagaccctgaggaaggccatgaaagggctcggcaccgatgaagacgccattattagcgtccttgcctaccgcaacaccgcccagcgccaggagatcaggacagcctacaagagcaccatcggcagggacttgatagacgacctgaagtcagaactgagtggcaacttcgagcaggtgattgtggggatgatgacgcccacggtgctgtatgacgtgcaagagctgcgaagggccatgaagggagccggcactgatgagggctgcctaattgagatcctggcctcccggacccctgaggagatccggcgcataagccaaacctaccagcagcaatatggacggagccttgaagatgacattcgctctgacacatcgttcatgttccagcgagtgctggtgtctctgtcagctggtgggagggatgaaggaaattatctggacgatgctctcgtgagacaggatgcccaggacctgtatgaggctggagagaagaaatgggggacagatgaggtgaaatttctaactgttctctgttcccggaaccgaaatcacctgttgcatgtgtttgatgaatacaaaaggatatcacagaaggatattgaacagagtattaaatctgaaacatctggtagctttgaagatgctctgctggctatagtaaagtgcatgaggaacaaatctgcatattttgctgaaaagctctataaatcgatgaagggcttgggcaccgatgataacaccctcatcagagtgatggtttctcgagcagaaattgacatgttggatatccgggcacacttcaagagactctatggaaagtctctgtactcgttcatcaagggtgacacatctggagactacaggaaagtactgcttgttctctgtggaggagatgattaaaataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaagattactttctactttgtgtttcacagacattgaatatattaaattattccatattttcttttcagtgaaaaattttttaaatggaagactgttctaaaatcacttttttccctaatccaatttttagagtggctagtagtttcttcatttgaaattgtaagcatccggtcagtaagaatgcccatccagttttctatatttcatagtcaaagccttgaaagcatctacaaatctctttttttaggttttgtccatagcatcagttgatccttactaagtttttcatgggagacttccttcatcacatcttatgttgaaatcactttctgtagtcaaagtataccaaaaccaatttatctgaactaaattctaaagtatggttatacaaaccatatacatctggttaccaaacataaatgctgaacattccatattattatagttaatgtcttaatccagcttgcaagtgaatggaaaaaaaaataagcttcaaactaggtattctgggaatgatgtaatgctctgaatttagtatgatataaagaaaacttttttgtgctaaaaatactttttaaaatcaattttgttgattgtagtaatttctatttgcactgtgcctttcaactccagaaacattctaagatgtacttggatttaattaaaaagttcactttgt

SEQ ID NO: 2 - protein, 319 aa, Homo sapiens
MATKGGTVKAASGFNAMEDAQTLRKAMKGLGTDEDAIISVLAYRNTAQRQEIRTAYKSTIGRDLIDDLKSELSGNFEQVIVGMMTPTVLYDVQELRRAMKGAGTDEGCLIEILASRTPEEIRRISQTYQQQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAEKLYKSMKGLGTDDNTLIRVMVSRAEIDMLDIRAHFKRLYGKSLYSFIKGDTSGDYRKVLLVLCGGDD

(19) United States
(12) Patent Application Publication
(10) Pub. No.: US 2011/0014270 A1
(43) Pub. Date: Jan. 20, 2011

(54) PREVENTION AND TREATMENT OF ISCHEMIA-REPERFUSION INJURY AND RELATED CONDITIONS

(75) Inventors: Vernon Michael Holers, Denver, CO (US); Liudmila Kulik, Aurora, CO (US); George C. Tsokos, Silver Springs, MD (US); Stephen Tomlinson, Mount Pleasant, SC (US)

Correspondence Address: SHERIDAN ROSS PC, 1560 BROADWAY, SUITE 1200, DENVER, CO, 80202, US

(73) Assignee: THE REGENTS OF THE UNIVERSITY OF COLORADO, Denver, CO (US)

(21) Appl. No.: 12/294,870

(22) PCT Filed: Mar. 27, 2007
(86) PCT No.: PCT/US07/65125

§ 371 Date: Oct. 1, 2010

Related U.S. Application Data
(60) Provisional application No. 60/786,527, filed on Mar. 27, 2006.

(51) Int. Cl. A61K 9/127; A61K 38/16; A61K 39/395; C07K 16/00; C07K 14/00; A61P 9/10; A61P 13/12; A61P 25/00; A61P 37/06

(52) U.S. Cl. 424/450; 514/15.1; 424/172.1; 530/389.1; 530/402

(57) ABSTRACT

Disclosed are Lipids, Annexin, and Lipid-Annexin Complexes for Use in the Prevention and/or Treatment of Ischemia-Reperfusion injury and reperfusion injury associated with a variety of diseases and conditions. Also disclosed are therapeutic targets and compositions for the prevention and treatment of ischemia-reperfusion injury and diseases and conditions associated with ischemia-reperfusion injury.

### FIELD OF THE INVENTION

This invention generally relates to the use of lipids, annexin, and lipid-annexin complexes for the prevention and/or treatment of ischemia-reperfusion injury and reperfusion injury associated with a variety of diseases and conditions, as well as therapeutic targets and compositions for the prevention and treatment of such diseases.

### BACKGROUND OF THE INVENTION

Ischemia-reperfusion (I-R) injury refers to damage to a tissue caused when the blood supply returns to the tissue after a period of ischemia (restriction in blood supply). The absence of oxygen and nutrients from the blood creates a condition in which the restoration of circulation results in inflammation and oxidative damage, rather than restoration of normal function. Ischemia-reperfusion injury can be associated with traumatic injury, including hemorrhagic shock, as well as many other medical conditions such as stroke or large vessel occlusion, and is a major medical problem. More particularly, ischemia-reperfusion injury is important in heart attacks, stroke, kidney failure following vascular surgery, post-transplantation injury and chronic rejection, as well as in various types of traumatic injury, where hemorrhage will lead to organ hypoperfusion, and then subsequent reperfusion injury during fluid resuscitation. Ischemia-reperfusion injury, or an injury due to reperfusion and ischemic events, is also observed in a variety of autoimmune and inflammatory diseases. Independently of other factors, ischemia-reperfusion injury leads to increased mortality.

Previous studies by the present inventors and colleagues have shown that certain types of natural antibodies recognize epitopes on ischemic tissue and catalyze the initiation and subsequent development of ischemia-reperfusion injury (Fleming et al., 2002, J. Immunol. 169:2126-2133; Rehrig et al., 2001, J. Immunol. 167:5921-5927). Ischemia-reperfusion injury, as well as hypovolemic shock and subsequent tissue damage, is known to be caused by complement and Fc receptor activation and the recruitment and activation of neutrophils and other inflammatory cells (Rehrig et al., 2001, supra). However, despite this understanding of the “downstream” mechanisms of tissue injury, the specific mechanism by which these pathogenic processes are initiated has, prior to the present invention, remained obscure.

Prior to the present invention, it had been shown that single monoclonal antibodies that react broadly with phospholipids and other extracellular or intracellular antigens such as DNA can cause ischemia-reperfusion injury in mice that lack other antibodies (i.e., B cell-deficient mice). Presumably, these antibodies can recognize determinants expressed on cells that are under stress during ischemia and that are beginning to undergo the early stages of apoptosis. However, despite such experiments that describe a possibility that natural antibodies might be important in ischemia-reperfusion injury, there has been no demonstration that shows this to be true in a setting where all other natural antibody types are present. Therefore, it has not been established whether the broad reactivity of any of the monoclonal antibodies was actually relevant to the ischemia-reperfusion injury disease process. Moreover, natural antibodies can recognize dozens of self and foreign antigens, and indeed in toto are believed to see every possible epitope that could be presented on a pathogen. Therefore, to date, there has not been a demonstration of a viable target for the development of therapeutic strategies that prevent or treat ischemia-reperfusion injury at the very early stages of the development of the condition.

Moreover, there is increasing evidence of reperfusion injury that can be found in autoimmune and inflammatory diseases that are not traditionally thought of as reperfusion injury-related. For example, the synovium in rheumatoid arthritis patients is a site that is subjected to constant reperfusion stress (e.g., low pH, lots of tissue pressure and poor perfusion). The higher quantity of synovial fluid found in hypermobile patients having this disease causes an increase in the intra-articular pressure, which is then exacerbated by joint motion. This may aggravate local inflammation through a hypoxic/reperfusion mechanism, which in turn causes oxidative injury due to intermittent ischemia (e.g., see Punzi et al., Rheumatology 2001; 40: 202-204; Pianon et al., Reumatismo 1996; 48(Suppl. 1):93; and Jawed et al., Ann Rheum Dis 1997; 56:686-9). A variety of inflammatory and autoimmune diseases can be associated with similar ischemic events.

Accordingly, there remains a need in the art to provide deliverable therapeutic agents and methods that prevent or reduce ischemia-reperfusion injury and reperfusion injury in an individual. For example, the successful development of an adjuvant therapy to fluid resuscitation, that can be given at the same time as the fluid resuscitation using the same delivery methods, would provide a substantial benefit to the individual who is at risk of or is developing ischemia-reperfusion injury. Similarly, the development of a therapy for use in patients suffering from chronic disease, including autoimmune disease and inflammatory conditions, that prevents damage associated with chronic and intermittent ischemia, would be valuable.

### SUMMARY OF THE INVENTION

One embodiment of the invention relates to a method to prevent or treat ischemia-reperfusion injury in an individual, comprising administering to the individual an agent that blocks or inhibits the binding of natural antibodies in the individual to: (a) annexin-4 expressed on the surface of a cell that is in or adjacent to a tissue that is undergoing, or is at risk of undergoing, ischemia-reperfusion injury; and/or (b) a phospholipid expressed on the surface of a cell that is in or adjacent to a tissue that is undergoing, or is at risk of undergoing, ischemia-reperfusion injury.

In one aspect, the phospholipid is selected from: phosphatidylcholine, phosphoglycerol, lysophosphatidylcholine, phosphatidic acid, phosphatidylethanolamine, phosphatidylserine, and a derivative of any of said phospholipids. In one aspect, the phospholipid is selected from: phosphatidylcholine and phosphoglycerol. In one aspect, the agent is a liposome or stable lipid moiety comprising said phospholipid, including, but not limited to, phospholipids consisting essentially of said phospholipid. In one aspect, the agent is a liposome or stable lipid moiety comprising a phospholipid selected from: phosphatidylcholine, phosphoglycerol, lysophosphatidylcholine, phosphatidic acid, phosphatidylethanolamine, phosphatidylserine, and a derivative of any of said phospholipids. In one aspect, the agent is a liposome or stable lipid moiety comprising a phospholipid selected from: phosphatidylcholine and phosphoglycerol, including, but not limited to a liposome or stable lipid moiety comprising phospholipids consisting essentially of phosphatidylcholine and phosphoglycerol. In one aspect, the liposome or stable lipid moiety comprises phosphotidylcholine, phosphoglycerol and cholesterol. In one aspect, the ratio of phosphotidylcholine:phosphoglycerol:cholesterol is 1:1:2.

In one aspect of this embodiment, the agent is an isolated annexin-4 protein or biologically active homologue thereof that binds to a phospholipid or comprises at least one conformational epitope bound by a natural antibody in the individual. In one aspect, the agent is an isolated annexin-4 protein.

In another aspect of this embodiment, the agent is an annexin-4-liposome complex or annexin-4-stable lipid moiety complex. For example, the liposome or stable lipid moiety portion of the complex can include, but is not limited to, a phospholipid selected from: phosphatidylcholine, phosphoglycerol, lysophosphatidylcholine, phosphatidic acid, phosphatidylethanolamine, phosphatidylserine, and a derivative of any of said phospholipids. In one aspect, the lipo some or stable lipid moiety portion of the complex comprises phospholipids consisting essentially of phosphatidylcholine and phosphoglycerol. In one aspect, the liposome or stable lipid moiety comprises phosphotidylcholine, phosphoglycerol and cholesterol.

In one aspect, the agent is a non-complement-fixing antibody or antigen-binding fragment thereof that selectively binds to annexin-4 and prevents the binding of a natural antibody to the annexin-4. In one aspect, the agent is a non-complement-fixing antibody or antigen-binding fragment thereof that blocks or inhibits the binding of a natural antibody to said phospholipid. In one aspect, the agent is a non-complement-fixing antibody or antigen-binding fragment thereof that blocks or inhibits the binding of a natural antibody to phosphotidylcholine and/or phosphoglycerol.

In another aspect, the agent is a drug that competitively inhibits the binding of a natural antibody to said phospholipid. In one aspect, the agent is a drug that competitively inhibits the binding of a natural antibody to phosphotidylcholine, phosphoglycerol or annexin-4.

In one aspect, the agent is a protein or polypeptide that competitively inhibits the binding of a natural antibody to said phospholipid. In one aspect, the agent is a protein or polypeptide that competitively inhibits the binding of a natural antibody to phosphotidylcholine, phosphoglycerol or annexin-4.

Another embodiment of the invention relates to a method to prevent or treat ischemia-reperfusion injury in an individual, comprising administering to the individual a liposome or stable lipid moiety comprising one or more phospholipids. In one aspect, the phospholipids are selected from: phosphatidylcholine, phosphoglycerol, lysophosphatidylcholine, phosphatidic acid, phosphatidylethanolamine, phosphatidylserine, and a derivative of any of said phospholipids. In another aspect, the phospholipids are selected from the group consisting of phosphatidylcholine and phosphoglycerol. In another aspect, the liposome or stable lipid moiety comprises phospholipids consisting essentially of phosphatidylcholine and phosphoglycerol. In another aspect, the liposome or stable lipid moiety comprises phosphotidylcholine, phosphoglycerol and cholesterol. In one aspect, the ratio of phosphotidylcholine:phosphoglycerol:cholesterol is 1:1:2.

Yet another embodiment of the invention relates to a method to prevent or treat ischemia-reperfusion injury in an individual, comprising administering to the individual an isolated annexin-4 protein or biologically active homologue thereof that binds to a phospholipid or comprises at least one conformational epitope bound by a natural antibody in the individual.

Another aspect of the invention relates to a method to prevent or treat ischemia-reperfusion injury in an individual, comprising administering to the individual an annexin-4-liposome complex, wherein the liposome portion of the complex comprises one or more phospholipids. In one aspect, the phospholipid is selected from: phosphatidylcholine, phosphoglycerol, lysophosphatidylcholine, phosphatidic acid, phosphatidylethanolamine, phosphatidylserine, and a derivative of any of said phospholipids. In one aspect, the phospholipid is selected from the group consisting of: phosphatidylcholine and phosphoglycerol.

In any of the above-described embodiments of the invention, in one aspect, the ischemia-reperfusion injury is catalyzed by natural antibodies in the individual. In one aspect, the ischemia-reperfusion injury is selected from: intestinal ischemia-reperfusion injury, renal ischemia-reperfusion injury, cardiac ischemia-reperfusion injury, ischemia-reperfusion injury of other internal organs such as the lung or liver, central nervous system ischemia-reperfusion injury, ischemia-reperfusion injury of the limbs or digits, or ischemia-reperfusion injury of any transplanted organ or tissue. In one aspect, the ischemia-reperfusion injury is associated with an autoimmune disease. In one aspect, the ischemia-reperfusion injury is associated with a disease or condition selected from: stroke, traumatic brain injury, spinal cord injury, trauma-induced hypovolemic shock, and rheumatoid arthritis.

In any of the above-described embodiments of the invention, in one aspect, the agent is administered with a pharmaceutically acceptable carrier. In one aspect, the agent is administered by a route selected from: nasal, inhaled, intratracheal, topical, and systemic route.

Yet another embodiment of the invention relates to a method to treat ischemia-reperfusion injury or a disease or condition associated with ischemia-reperfusion injury in an individual. The method includes administering to the individual a therapeutic agent for the treatment of ischemia-reperfusion injury or a disease or condition associated with ischemia-reperfusion injury, wherein the agent is linked to a targeting agent that selectively binds to annexin-4 expressed on the surface of a cell that is in or adjacent to a tissue that is undergoing, or is at risk of undergoing, ischemia-reperfusion injury, or to a phospholipid expressed on the surface of a cell that is in or adjacent to a tissue that is undergoing, or is at risk of undergoing, ischemia-reperfusion injury. In one aspect, the targeting agent is an antibody or antigen-binding fragment thereof, wherein the antibody is a competitive inhibitor of an antibody that selectively binds to annexin-4 expressed on the surface of a cell or to a phospholipid expressed on the surface of a cell and that catalyzes the initiation and development of ischemia-reperfusion injury. In one aspect, the targeting agent is an antigen-binding fragment or non-pathogenic form of an antibody that selectively binds to annexin-4 expressed on the surface of a cell or to a phospholipid expressed on the surface of a cell and that catalyzes the initiation and development of ischemia-reperfusion injury.

Another embodiment of the invention relates to the use of an agent in the preparation of a medicament for the prevention or treatment of ischemia-reperfusion injury, wherein the agent blocks or inhibits the binding of natural antibodies in the individual to: (a) annexin-4 expressed on the surface of a cell that is in or adjacent to a tissue that is undergoing, or is at risk of undergoing, ischemia-reperfusion injury; and/or (b) a phospholipid expressed on the surface of a cell that is in or adjacent to a tissue that is undergoing, or is at risk of undergoing, ischemia-reperfusion injury.

Another embodiment of the invention relates to the use of a liposome or stable lipid moiety consisting essentially of phosphotidylcholine, phosphoglycerol and cholesterol in the preparation of a medicament for the prevention or treatment of ischemia-reperfusion injury.

Yet another embodiment of the invention relates to the use of an annexin-4 liposome complex, wherein the liposome comprises at least one phospholipid in the preparation of a medicament for the prevention or treatment of ischemia-reperfusion injury.

Another embodiment of the invention relates to the use of an agent in the preparation of a medicament for the treatment of an autoimmune disease, wherein the agent blocks or inhibits the binding of natural antibodies in the individual to: (a) annexin-4 expressed on the surface of a cell; and/or (b) a phospholipid expressed on the surface of a cell.

Another embodiment of the invention relates to use of a liposome or stable lipid moiety consisting essentially of phosphotidylcholine, phosphoglycerol and cholesterol in the preparation of a medicament for the treatment of an autoimmune disease.

Yet another embodiment of the invention relates to the use of an annexin-4 liposome complex, wherein the liposome comprises at least one phospholipid in the preparation of a medicament for the treatment of autoimmune disease.

Another embodiment of the invention relates to an isolated antibody that selectively binds to annexin-4, wherein the antibody induces ischemia-reperfusion injury in an animal. In one aspect, the antibody is MAb B4.

Yet another embodiment of the invention relates to an isolated antibody that selectively binds annexin-4, wherein the antibody competitively inhibits the binding of the antibody of the above-described antibody that selectively binds to annexin-4, to annexin-4, and wherein the antibody does not induce ischemia-reperfusion injury in an animal.

Another embodiment of the invention relates to an isolated antibody that selectively binds to at least one phospholipid, wherein the antibody induces ischemia-reperfusion injury in an animal. In one aspect, the antibody is MAb C2.

Yet another embodiment of the invention relates to an isolated antibody that selectively binds to at least one phospholipid, wherein the antibody competitively inhibits the binding of the above-described antibody that selectively binds to at least one phospholipid, to the phospholipid, and wherein the antibody does not induce ischemia-reperfusion injury in an animal.

Another embodiment of the invention relates to a liposome or stable lipid moiety that inhibits ischemia-reperfusion injury in an individual, wherein the liposome or stable lipid moiety consists essentially of phosphotidylcholine, phosphoglycerol and cholesterol. In one aspect, the ratio of phosphotidylcholine:phosphoglycerol:cholesterol is 1:1:2.

Yet another embodiment of the invention relates to an annexin-4 liposome complex, wherein the liposome comprises at least one phospholipid. In one aspect, the phospholipid is selected from: phosphatidylcholine, phosphoglycerol, lysophosphatidylcholine, phosphatidic acid, phosphatidylethanolamine, phosphatidylserine, and a derivative of any of said phospholipids. In one aspect, the phospholipid is selected from: phosphotidylcholine and phosphoglycerol. In one aspect, the liposome comprises phospholipids consisting essentially of phosphatidylcholine and phosphoglycerol. In one aspect, the liposome comprises phosphotidylcholine, phosphoglycerol and cholesterol. In one aspect, the ratio of phosphotidylcholine:phosphoglycerol:cholesterol is 1:1:2.

Yet another embodiment of the invention relates to a method to treat an autoimmune disease, comprising administering to the individual an agent that blocks or inhibits the binding of natural antibodies in the individual to: (a) annexin-4 expressed on the surface of a cell that is in or adjacent to a tissue that is undergoing, or is at risk of undergoing, ischemia-reperfusion injury; and/or (b) a phospholipid expressed on the surface of a cell that is in or adjacent to a tissue that is undergoing, or is at risk of undergoing, ischemia-reperfusion injury. In one aspect, the autoimmune disease is rheumatoid arthritis. In one aspect, the agent is a liposome or stable lipid moiety comprising said phospholipid. In one aspect, the agent is a liposome or stable lipid moiety comprising a phospholipid selected from: phosphatidylcholine, phosphoglycerol, lysophosphatidylcholine, phosphatidic acid, phosphatidylethanolamine, phosphatidylserine, and a derivative of any of said phospholipids. In one aspect, the agent is a liposome or stable lipid moiety comprising a phospholipid selected from: phosphatidylcholine and phosphoglycerol. In one aspect, the agent is an isolated annexin-4 protein or biologically active homologue thereof that binds to a phospholipid or comprises at least one conformational epitope bound by a natural antibody in the individual. In one aspect, the agent is an annexin-4-liposome complex or annexin-4-stable lipid moiety complex.

### BRIEF DESCRIPTION OF THE DRAWINGS OF THE INVENTION

FIG. 1 is a graph showing various monoclonal antibodies readily transfer the capacity of Rag−/− mice to develop intestinal ischemia-reperfusion injury.

FIG. 2 is a graph showing liposomes composed of cholesterol and the phospholipids phosphatidylcholine and phosphoglycerol can nearly completely block the development of intestinal ischemia-reperfusion injury in immune-competent, C57B/6 mice.

FIG. 3 is a schematic drawing showing the chemical structures of phosphatidylcholine and phosphoglycerol.

FIG. 4 is a graph showing that when subjected to MCAO-induced ischemia and 24 h of reperfusion, Rag1−/− (n=18) survived to the primary end point of 24 h post reperfusion, whereas C57BL/6 mice (n=12) had only a 59% survival rate (p<0.001).

FIG. 5 is a graph showing that Rag1−/− significantly protects against cerebral infarct when compared to controls (*p=0.001), and that reconstitution with monoclonal antibodies C2 and B4, but not D5, induce cerebral infarct in a dose dependant manner.

FIG. 6 is a graph showing a significant reduction in neurological deficit associated with Rag1−/− when compared to wildtype (*p=0.03), and that higher doses of both C2 and B4 monoclonal antibodies show a trend towards a poorer neurological outcome post stroke.

FIG. 7 is a graph showing that administration of recombinant annexin IV significantly protects against cerebral infarct when compared to controls.

FIG. 8 is a graph showing a significant reduction in neurological deficit after administration of recombinant annexin IV during brain ischemia-reperfusion injury.

FIG. 9 is a graph showing that injection of recombinant annexin IV reduces the level of intestinal ischemia reperfusion injury in C57Bl/6 mice.

FIG. 10 is a graph showing that administration of an anti-annexin IV monoclonal antibody significantly worsens arthritic symptoms in a model of rheumatoid arthritis.

### DETAILED DESCRIPTION OF THE INVENTION

This present invention is generally related to novel therapeutic agents and methods for the prevention and/or treatment of ischemia-reperfusion (I-R or I/R) injury, which includes reperfusion injury in a variety of diseases and conditions. The present inventors provide herein new therapeutics that target the very first steps of reperfusion injury and that can be used alone, or in conjunction with fluids or other therapeutic strategies, to provide a major benefit and substantially decrease the morbidity and mortality associated with injuries and conditions that may result in ischemia-reperfusion injury. The present inventors also provide herein targets that can be used to efficiently direct therapeutic modalities to a site of reperfusion injury.

First, the present inventors show for the first time that one class of antigenic epitopes recognized by natural antibodies, defined operationally in the inventors' experiments as being expressed on liposomes composed of cholesterol and the phospholipids, phosphatidylcholine and phosphoglycerol (although the invention is not limited to these phospholipids), are required for the development of ischemia-reperfusion injury in mice with an intact and normal natural antibody immune repertoire. Specifically, the present inventors have demonstrated that infusion of liposomes composed of cholesterol and phosphatidylcholine and phosphoglycerol, either systemically or into the intestinal lumen, blocks the development of intestinal ischemia-reperfusion injury in immune competent mice. Similar results have also been achieved in a model of cerebral injury following ischemic stroke. Thus, of all of the potential targets of pathogenic natural antibodies, the epitopes displayed on this liposome are essential for reperfusion injury. The inventors' data supports the hypothesis that other lipid-binding proteins can also serve as targets for pathogenic natural antibodies in ischemia-reperfusion and hemorrhagic shock. The present inventors show for the first time herein that ischemia-reperfusion injury in an immune competent individual can be blocked using liposomes bearing a small subset of phospholipids.

Second, the present inventors have identified a new antigen recognized by a novel anti-protein monoclonal antibody that catalyzes ischemia-reperfusion injury. This monoclonal antibody is derived from the natural antibody repertoire, is pathogenic in mice lacking any antibodies (B cell-deficient mice), and specifically recognizes the phospholipid binding protein, annexin-4 (also referred to herein as annexin IV). The inventors have also demonstrated that administration of annexin-4 (e.g., as a recombinant protein) inhibits the development of intestinal ischemia-reperfusion injury, as well as cerebral injury following ischemic stroke. In addition, the inventors have demonstrated that an annnexin-4 antibody is pathogenic in a model of rheumatoid arthritis (CIA-induced arthritis), demonstrating that the same therapeutic strategies described herein that have been applied to more conventional types of ischemia-reperfusion injury can also be applied to a wide variety of autoimmune and inflammatory diseases wherein chronic or intermittent bouts of reperfusion injury and ischemic events damage tissues and cells.

In total, the present inventors have shown that inhibition of the reactivity of natural antibodies that recognize cell surface phospholipids and/or the phospholipid binding protein annexin-4, all of which comprise epitopes expressed on cells that are undergoing apoptosis, blocks the development of ischemia-reperfusion injury in a variety of conditions and diseases. These novel discoveries have lead the present inventors to set forth herein a method of using agents that interrupt the development of this catastrophic injury at its earliest point by blocking natural antibody recognition of ischemia-induced targets. Blockade at this early point can limit the activation of the wide array of “downstream” pro-inflammatory pathways and, with one targeted therapeutic, achieve a broad inhibition of many pathways.

Accordingly, one embodiment of the present invention relates to compositions comprising at least one agent that prevents or inhibits ischemia-reperfusion injury in an individual by blocking or inhibiting (reducing) the interaction of natural antibodies in an individual with: (1) annexin-4 expressed on the surface of a cell that is in or adjacent to a tissue that is undergoing ischemia-reperfusion injury (or is at risk of undergoing ischemia-reperfusion injury); and/or (2) a phospholipid expressed on the surface of a cell that is in or adjacent to a tissue that is undergoing ischemia-reperfusion injury (or is at risk of undergoing ischemia-reperfusion injury). The phospholipids can include, but are not limited to, phosphatidylcholine, phosphoglycerol, lysophosphatidylcholine, phosphatidic acid, phosphatidylethanolamine, phosphatidylserine, and derivatives of any of such phospholipids (e.g., polyethylene glycol (PEG) conjugates of these phospholipids, such as phosphatidylethanolamine-PEG). In one preferred embodiment, the phospholipids are selected from phosphatidylcholine and/or phosphoglycerol and/or derivatives thereof. Under conditions that induce ischemia-reperfusion injury, such a cell is typically stressed or in the early stages of apoptosis (described in more detail below).

An agent useful in the present invention can be any agent that blocks or inhibits the interaction of a natural antibody in an individual with the above-described molecules, including, but not limited to, liposomes and other lipid moieties as described herein, soluble proteins or polypeptides, non-complement activating antibodies or antigen-binding fragments thereof, or small molecules (e.g. synthetic compounds or drugs). Included in the invention are the use of the antigen-combining sites identified herein by the antibodies of the invention or the sites of the antigen bound by such antibodies, to direct any therapeutics to the sites of antigens that are bound by natural antibodies and these antibodies, for the prevention and treatment of ischemia-reperfusion injury.

In one preferred embodiment of the present invention, an agent useful in the present invention is selected from: (1) a liposome or other lipid moiety comprising phospholipids (e.g., phosphatidylcholine, phosphoglycerol, lysophosphatidylcholine, phosphatidic acid, phosphatidylethanolamine, and/or phosphatidylserine, and/or derivatives of any of such phospholipids); (2) an isolated annexin-4 protein or biologically active homologue thereof; and/or (3) an annexin-4-liposome complex, wherein the liposome portion of the complex comprises the phospholipids as described in (1) above. In one embodiment, the phospholipids contained in the liposomes or lipid moieties described herein consist essentially of or consist of the phospholipids selected from phosphatidylcholine, phosphoglycerol, lysophosphatidylcholine, phosphatidic acid, phosphatidylethanolamine, and/or phosphatidylserine, and/or derivatives of any of such phospholipids, with the phospholipids, phosphatidylcholine and/or phosphoglycerol being one preferred embodiment. In one embodiment, any of the above-described liposomes or lipid moieties further comprise cholesterol or any other lipid or lipid derivative that is useful for stabilizing the bilayer of lipids in a liposome and/or decreasing leakage of encapsulated material. In one embodiment, any of the above-described liposomes further comprise antioxidants such as α-tocopherol or β-hydroxytoluidine. Such antioxidants are useful for inhibiting oxidation of the lipids in liposomes.

In one embodiment of the present invention, the agent is a non-complement-fixing antibody or antigen-binding fragment thereof that selectively binds to annexin-4 and prevents the binding of a natural antibody to the annexin-4. In another embodiment of the present invention, the agent is a non-complement-fixing antibody or antigen-binding fragment thereof that blocks or inhibits the binding of a natural antibody to phospholipids including phosphotidylcholine and/or phosphoglycerol. Other compounds that competitively inhibit the binding of natural antibodies to any of annexin-4, phosphotidylcholine and/or phosphoglycerol (or other phospholipids targeted by natural antibodies and associated with reperfusion injury) will be apparent to those of skill in the art given the present disclosure. The present invention also relates to the use of any of the compositions described herein for the prevention or treatment of ischemia-reperfusion injury.

Another embodiment of the present invention relates generally to an antibody, antigen-binding fragment thereof, or antigen-binding peptide that selectively binds to annexin-4, wherein the antibody antigen-binding fragment thereof, or antigen-binding peptide is capable of inducing ischemia-reperfusion injury in an immune incompetent host. Such an antibody antigen-binding fragment thereof, or antigen-binding peptide is useful in an animal model of ischemia-reperfusion injury, where such model enables the development of therapeutic agents and methods for the prevention or treatment of ischemia-reperfusion injury. Such antibodies, antigen-binding fragment thereof, or antigen-binding peptides are also useful for identifying competitive inhibitors of natural antibodies and any of the inhibitors described above, for example, and in modified forms (e.g., non-pathogenic forms including fragments thereof) for targeting therapeutic moieties to a site for the prevention or inhibition of ischemic-reperfusion injury or for developing therapeutic moieties that target the same antigen-combining sites as these antibodies (i.e., targeting to the sites of injury). Antibodies the competitively inhibit the binding of such an antibody to annexin-4 are also encompassed by the invention.

As discussed above, one composition or agent useful in the present invention is generally described as an agent that prevents or inhibits ischemia-reperfusion injury in an individual by blocking or inhibiting (reducing, decreasing) the interaction of natural antibodies with one or more of the targets described above (i.e., annexin-4, or phospholipids, such as phosphatidylcholine and/or phosphoglycerol), where the targets are expressed on the surface of a cell that is in or adjacent to a tissue that is undergoing ischemia-reperfusion injury, or that is at risk of undergoing ischemia-reperfusion injury (e.g., a tissue that has been subjected to trauma or disease and is ischemic or under ischemic conditions). Another composition or agent useful in the present invention directs or targets a therapeutic moiety to the site of potential or realized ischemia-reperfusion injury in an individual by binding to the same or similar sites as the natural antibodies or the pathogenic antibodies described herein.

According to the present invention, a “natural antibody” is an antibody that exists in an immune competent individual (i.e., an individual that has an intact or normal immune system, and particularly, an intact B cell compartment), where the antibody has been produced in the individual without any evidence of prior contact with the specific antigen (i.e., there is no identifiable immunogenic origin of the antibody). In other words, a natural antibody is an antibody that can be found in the serum or plasma of an individual not known to have been stimulated by the specific antigen to which the antibody binds, either artificially or as the result of naturally occurring contact (e.g., an infection). Natural antibodies are typically fairly low affinity antibodies and may be polyreactive (i.e., react with or bind to more than one antigen), and are usually of the IgM or IgG isotype. However, some natural antibodies, for example MAb B4 as described below, can be highly specific for an antigen (e.g., in this exemplary case, annexin IV). This invention describes natural antibodies with features of both types, and specifically, antibodies that react with subsets of phospholipids and antibodies that react specifically with one antigen. The meaning of the term “natural antibody” is well-known to those of skill in the art.

Ischemia-reperfusion injury is a well-known condition in the art. As described above, ischemia-reperfusion injury generally refers to damage to a tissue caused when the blood supply returns to the tissue (reperfusion) after a period of ischemia (restriction in blood supply). The absence of oxygen and nutrients from the blood creates a condition in which the restoration of circulation results in inflammation and oxidative damage, rather than restoration of normal function. Ischemia-reperfusion injury can cause increases in the production of or oxidation of various potentially harmful compounds produced by cells and tissues, as well as inflammation, which can lead to oxidative damage to and/or death of cells and tissues. For example, renal ischemia-reperfusion injury can result in histological damage to the kidneys, including kidney tubular damage and changes characteristic of acute tubular necrosis. The resultant renal dysfunction permits the accumulation of nitrogenous wastes ordinarily excreted by the kidney, such as serum urea nitrogen (SUN). Ischemia-reperfusion may also cause injury to remote organs, such as the lung, and is associated with a wide variety of diseases and conditions involving inflammation and/or autoimmunity, for example.

An ischemia-reperfusion injury that can be prevented or treated according to the present invention includes any injury due to one or more ischemic events and reperfusion that occurs in any organ or tissue and in the context of a healthy individual or in any disease or condition. Ischemia-reperfusion injuries include, but are not limited to, intestinal ischemia-reperfusion injury, renal ischemia-reperfusion injury, cardiac ischemia-reperfusion injury, ischemia-reperfusion injury of other internal organs such as the lung or liver, central nervous system ischemia-reperfusion injury, ischemia-reperfusion injury of the limbs or digits, trauma-induced hypovolemia, or ischemia-reperfusion injury of any transplanted organ or tissue. Ischemia-reperfusion injury can also occur in conjunction with a variety of other conditions including, but not limited to, stroke, traumatic brain injury, spinal cord injury, trauma-induced hypovolemic shock, and autoimmune diseases such as rheumatoid arthritis (e.g., which can be greatly worsened by ischemic injury of the synovium) or a variety of other inflammatory diseases (diseases mediated by inflammation or wherein inflammation is a symptom that may result in or be associated with ischemic events and reperfusion). Other conditions and diseases in which ischemia-reperfusion injury occurs will be known to those of skill in the art.

Autoimmune diseases that can be treated by the invention, include, but are not limited to, rheumatoid arthritis, systemic lupus erythematosus, multiple sclerosis, myasthenia gravis, insulin-dependent diabetes mellitus, acute disseminated encephalomyelitis, Addison's disease, antiphospholipid antibody syndrome, autoimmune hepatitis, Crohn's disease, Goodpasture's syndrome, Graves' disease, Guillain-Barré syndrome, Hashimoto's disease, idiopathic thrombocytopenic purpura, pemphigus, Sjögren's syndrome, and Takayasu's arteritis.

Viral infections are also frequently associated with inflammation and may lead to ischemic events and ischemia-reperfusion injury. Inflammation due to infection by bacteria, parasites, fungi and protozoa, are also included as potential targets for the invention.

According to the present invention, a cell that is in or adjacent to a tissue that is at undergoing ischemia-reperfusion injury or is at risk of undergoing ischemia-reperfusion injury, includes a cell that is part of a tissue or organ, or adjacent to (near, directly next to, in the microenvironment of, bordering, flanking, adjoining) a tissue or organ, in which ischemia-reperfusion injury is going to occur, is likely to occur, or is beginning to occur. In the case of an adjacent cell, the cell is sufficiently within the microenvironment of the ischemic tissue or organ such that conditions of oxidative damage and/or inflammation affect the adjacent cell, as well as the ischemic tissue or organ. Such a cell may display signs of stress, including, but not limited to, the display of “stress proteins” (e.g., heat shock proteins and other proteins associated with a cellular stress response, including annexins) or other molecules on the cell surface (phospholipids, carbohydrate moieties), including the display of abnormal levels of proteins or other molecules on the cell surface. Such a cell may be undergoing apoptosis or showing signs of apoptosis, such signs including morphological changes in the cell, chromatin condensation, changes in cellular signal transduction protein interactions, changes in intracellular calcium levels, externalization of phospholipids, cell detachment, loss of cell surface structures, etc.

As discussed above, it has been previously demonstrated that natural antibodies recognize epitopes on ischemic tissue and catalyze the initiation and subsequent development of ischemia-reperfusion injury (1,2). The present inventors have now demonstrated that, out of the incredibly large number of possible epitopes to which a natural antibody could bind, the epitopes displayed on the exemplary lipids described herein, composed of phosphatidylcholine, phosphoglycerol and cholesterol, are essential for ischemia-reperfusion injury, in that blocking the binding of these epitopes by natural antibodies can prevent ischemia-reperfusion injury. In addition, annexin-4 has also been shown by the present inventors herein to be a novel target for blocking or inhibiting ischemia-reperfusion injury. All of these compounds are known to be expressed on cells undergoing apoptosis. The present inventors intend that the present invention be extended to other lipids, including other phospholipids than phosphatidylcholine and phosphoglycerol, including, but not limited to, lysophosphatidylcholine, phosphatidic acid, phosphatidylethanolamine, and/or phosphatidylserine, and/or derivatives of any of such phospholipids.

Accordingly, one embodiment of the invention relates to agents or compositions that inhibit the binding of phospholipids (e.g., phosphatidylcholine, phosphoglycerol, lysophosphatidylcholine, phosphatidic acid, phosphatidylethanolamine, and/or phosphatidylserine, and/or derivatives of any of such phospholipids) by a natural antibody in an individual. Such an agent can include, but is not limited to, liposomes or lipid moieties comprising, consisting essentially of, or consisting of, one or more phospholipids. Preferred phospholipids include, but are not limited to, phosphatidylcholine, phosphoglycerol, lysophosphatidylcholine, phosphatidic acid, phosphatidylethanolamine, and/or phosphatidylserine, and/or derivatives of any of such phospholipids, with phosphatidylcholine and/or phosphoglycerol being one preferred set of phospholipids. Such liposomes competitively inhibit the binding of natural antibodies to the phospholipids on the surface of cells in or adjacent to ischemic tissues or organs. Such an agent can also include, but is not limited to, antibodies or antigen-binding fragments thereof that do not fix complement (non-complement fixing antibodies) and that selectively bind to the phospholipids; other lipid-containing moieties that competitively inhibit the binding of natural antibodies to the phospholipids on the surface of cells in or adjacent to ischemic tissues or organs (e.g., any stable lipid formulation); a protein or polypeptide that competitively inhibits the binding of natural antibodies to the phospholipids on the surface of cells in or adjacent to ischemic tissues or organs; or a small molecule that inhibits the binding of natural antibodies to the phospholipids on the surface of the cells. In a preferred embodiment, the agent includes liposomes or stable lipid moieties comprising, consisting essentially of, or consisting of one or more phospholipids, with phospholipids selected from one or more of phosphatidylcholine, phosphoglycerol, lysophosphatidylcholine, phosphatidic acid, phosphatidylethanolamine, and/or phosphatidylserine, and/or derivatives of any of such phospholipids being particularly preferred, wherein the liposomes competitively inhibit the binding of natural antibodies to the phospholipids on the surface of cells (and particularly stressed or apoptotic cells) in or adjacent to ischemic tissues or organs.

Another embodiment of the invention relates to agents or compositions that inhibit the binding of natural antibodies to an annexin protein, and particularly, to annexin-4. Such an agent can include, but is not limited to, a non-complement fixing antibody that selectively binds to annexin-4; a soluble annexin protein or homologue thereof (preferably annexin-4) that competitively inhibits the binding of natural antibodies to the annexin on the surface of cells in or adjacent to ischemic tissues or organs; a phospholipid that that competitively inhibits the binding of natural antibodies to the annexin on the surface of cells in or adjacent to ischemic tissues or organs; or a small molecule that inhibits the binding of natural antibodies to the annexin on the surfaces of cells, and particularly, stressed or apoptotic cells. In one preferred embodiment, the agent comprises an annexin protein (and preferably annexin-4) or homologue thereof complexed with a liposome that comprises one or more phospholipids as discussed above.

A preferred embodiment of the invention relates to a liposome or other stable lipid moiety comprising lipids including, consisting essentially of, or consisting of, one or more phospholipids. In one embodiment, the phospholipids are selected from one or more of phosphatidylcholine (PC), phosphoglycerol (PG), lysophosphatidylcholine, phosphatidic acid, phosphatidylethanolamine (PE), and/or phosphatidylserine (PS), and/or derivatives of any of such phospholipids (e.g., a PEG derivative, such as phosphatidylethanolamine-PEG). In one preferred embodiment, the phospholipids are selected from phosphatidylcholine (PC) and/or phosphoglycerol (PG) and/or derivatives thereof. In another embodiment, the liposomes or other stable lipid moiety include another lipid, such as cholesterol, or any other lipid or lipid derivative that is useful for stabilizing the bilayer of lipids in a liposome and/or decreasing leakage of encapsulated material. In one embodiment, any of the liposomes or lipid moieties described herein further comprise antioxidants such as α-tocopherol or β-hydroxytoluidine. Such antioxidants are useful for inhibiting oxidation of the lipids in liposomes. In one exemplary embodiment, the liposome or lipid moiety is composed of PC, PG, and cholesterol, although other liposome and lipid compositions are contemplated, including pure PC, pure PG, combinations of PC and PG, combinations of PC and cholesterol, combinations of PG and cholesterol, and other lipid compositions including PC and/or PG and at least one other lipid. Similarly, liposomes and lipid moieties useful herein can be composed of any one or combination of a different phospholipid, including any specifically described herein, and in combination cholesterol or another lipid(s).

According to the present invention, a liposome is a spherical, microscopic artificial membrane vesicle consisting of an aqueous core enclosed in one or more phospholipid layers. Liposomes can also be generally defined as self closed spherical particles with one or several lipid membranes. Liposomes can be composed of naturally-derived phospholipids with mixed fatty acid chains or prepared from synthetic lipids with well-defined lipid chains. Depending on the number of the membranes and size of the vesicles, liposomes are considered to be large multilamellar vesicles (LMV) with sizes up to 500 nm, small unilamellar vesicles (SUV) with sizes <100 nm, and large unilamellar vesicles (LUV) with sizes >100 nm. Liposomes and liposome preparation methods are well known in the art, and one example of liposomes useful in the present invention, as well as a method of producing such liposome, is described in the Examples. Other lipid moieties include any lipid composition comprising or containing the lipids described herein that is stable for use in a method of the invention. For example, non-liposomal lipids can be stabilized by the use of a lipoprotein (e.g., see Nanodisc™, Nanodisc, Inc.).

In one aspect of the invention, when the liposome or lipid moiety comprises PC, PG and cholesterol, the molar ratio of lipids within the liposome or lipid moiety can range from 1:1:1 (PC:PG:cholesterol) to 20:1:1 to 1:20:1 to 1:1:2. Some preferred ratios include, but are not limited to 2:1:2, 4:1:2, 6:1:2, 1:2:2, 1:4:2, 1:6:2 with a ratio of 1:1:2 being one preferred ratio. The molar ratio of PC:PG can range from 1:20 to 20:1 in any liposome or lipid moiety where both phospholipids are included. In embodiments where only one of PC or PG is included with at least one other lipid (e.g., cholesterol), the molar ratio of either PC or PG to the other lipid(s) can range from 20:1 to 1:20. In embodiments where other phospholipid combinations are used, the molar ratio of any one phospholipid to another phospholipid in the liposome can range from 20:1 to 1:20.

The total concentration of lipids to be included in a liposome or lipid moiety useful in the present invention can range from about 5 μmol to about 100 μmol per 1 ml of liposomes, including any amount between, in increments of 1 μmol. When the phospholipids are the only component of the liposome, one preferred concentration of phospholipids is about 44 μmol per 1 ml of liposomes. When cholesterol and phospholipids are included in the liposome or lipid moiety, one preferred concentration of lipids is about 88 μmol per 1 mol of liposomes or lipid moiety.

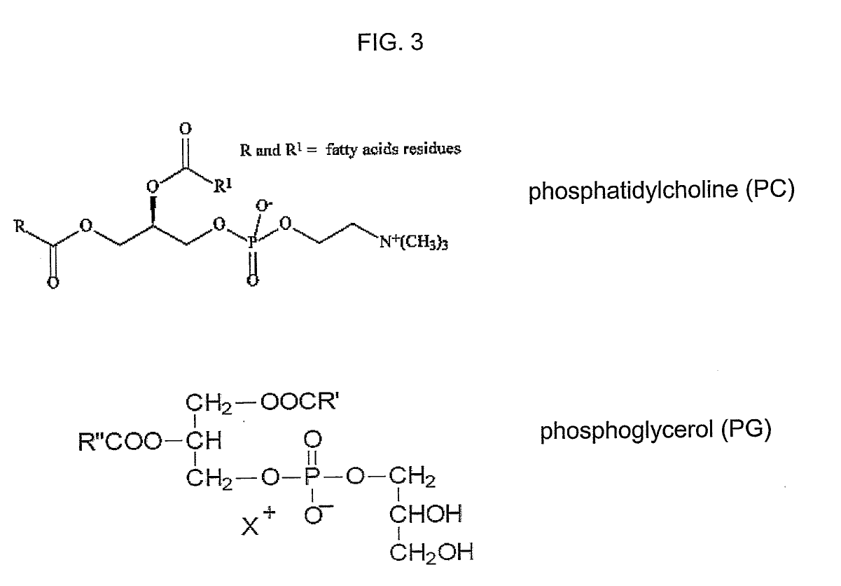

Phosphatidylcholine (PC) is a polar lipid that is a major constituent of cell membranes. PC is also known as 1,2-diacyl-:ussn:ue-glycero-3-phosphocholine, PtdCho and lecithin (when used in the chemical sense). The structure of PC is represented in FIG. 3. The fatty acid composition of PC from plant and animal sources differ. For example, the saturated fatty acids, such as palmitic acid and stearic acid, make up 19 to 24% of soya PC, the monounsaturated oleic acid contributes 9 to 11%, linoleic acid provides 56 to 60%, and alpha-linolenic acid makes up 6 to 9%. In egg yolk PC, the saturated fatty acids, palmitic acid and stearic acid, make up 41 to 46% of egg PC, oleic acid makes up 35 to 38%, linoleic acid 15 to 18%, and alpha-linolenic 0 to 1%. PC is important for normal cellular membrane composition and repair, and is the major delivery form of the essential nutrient, choline. Choline itself is a precursor in the synthesis of the neurotransmitter acetylcholine, the methyl donor betaine and phospholipids, including PC and sphingomyelin among others.

Phosphoglycerol (PG) is a ubiquitous phospholipid that is a major component of bacterial cell membranes and a lesser component of animal and plant cell membranes. In animal cells, PG may serve primarily as a precursor for diphosphatidylglycerol (cardiolipin). PG is the second most abundant phospholipid in lung surfactant in most animal species. The generic structure of PG is represented in FIG. 3.

The structure of other phospholipids described herein are also well-known in the art, as well as the roles of such phospholipids in biological systems.

Cholesterol is an essential constituent of plasma membranes in mammalian cells found in many biomembranes at very high concentration (Yeagle, 1993. The Membranes of Cells, 2nd Ed. Academic Press, San Diego; Yeagle, 1985, Biochim. Biophys. Acta. 822:267-287; Gennis, 1989. Biomembranes: Molecular Structure and Function. Springer-Verlag, New York). It has a pronounced effect on the physical properties of membranes, particularly on the structure of the phospholipid bilayers (Yeagle et al., 1977, Biochemistry. 20:4344-4349; Forbes et al., 1988, J. Am. Chem. Soc. 110:1059-1065; Sankaram and Thompson, 1990, Biochemistry. 29:10676-10675; Mukherjee and Chattopadhyay, 1996, Biochemistry. 35:1311-1322; DuFourc et al., 1984, Biochemistry. 23:6062-6071; Robinson et al., 1995, Biophys. J. 68:164-170; Lasic, 1993, Liposomes: From Physics to Applications. Elsevier, New York. 201-108). The effect of cholesterol on transport kinetics across liposome bilayers is important to the application of liposomes as drug delivery systems because cholesterol is often added to optimize the permeability of the liposome bilayers (Janoff, 1999, Liposomes: Rational Design. Marcel Dekker, New York; Lasic and Papahadjopoulos, 1998, Medical Applications of Liposomes. Elsevier, New York).

In one embodiment, the liposome or lipid moiety useful in the present invention is complexed with another agent, such as a protein or a small molecule (drug), wherein the other agent is also useful for inhibiting or preventing ischemia-reperfusion injury in an individual or treating an aspect of a disease or condition in which ischemia-reperfusion injury is occurring, may occur, or has occurred. In one embodiment, the liposomes or lipid moieties of the present invention are complexed with annexin-4, including with a fragment or homologue of annexin-4 (described below). Methods of encapsulating or complexing proteins and other agents with liposomes are known in the art. The encapsulation efficiency of proteins by liposomes generally depends on interaction between the protein and the lipid bilayer. The protein entrapment can be increased by manipulation of the liposomal lipid composition, or by increasing the lipid concentration, in order to favor electrostatic interactions, while monitoring the ionic strength of the protein solution (Colletier et al., BMC Biotechnology 2002, 2:9). In the case of annexin-4, because the protein binds to phospholipids, conventional entrapment methods are likely not to be necessary, and the annexin-4 may be complexed with the liposomes simply by mixing recombinantly produced annexin-4 with the liposomes or lipids used to form the liposomes. Preferably, the amount of annexin-4 complexed with liposomes will range from about 0.001 mg of annexin-4 protein per 1 ml liposome to about 5 mg of annexin-4 protein per 1 ml liposomes.

Another embodiment of the invention relates to an annexin-4 protein for use in the present invention. In one embodiment, the annexin-4 protein is provided as a protein, fragment thereof or homologue thereof, and is used as a therapeutic agent. The annexin-4 protein can be used alone or in a composition. One composition comprises a liposome as described herein that is complexed with the annexin-4 protein. The annexin-4 protein or a suitable portion thereof (i.e., forming at least one conformational epitope for antibody binding) can also be used to produce antibodies according to the present invention.

An isolated protein, according to the present invention, is a protein that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein of the present invention is produced recombinantly. Reference to a particular protein from a specific organism, such as a “human annexin-4 protein”, by way of example, refers to an annexin-4 protein (including a homologue of a naturally occurring annexin-4 protein) from a human or an annexin-4 protein that has been otherwise produced from the knowledge of the structure (e.g., sequence) of a naturally occurring annexin-4 protein from a human. In other words, a human annexin-4 protein includes any annexin-4 protein that has the structure and function of a naturally occurring annexin-4 protein from a human or that has a structure and function that is sufficiently similar to a human annexin-4 protein such that the annexin-4 protein is a biologically active (i.e., has biological activity) homologue of a naturally occurring annexin-4 protein from a human. As such, a human annexin-4 protein can include purified, partially purified, recombinant, mutated/modified and synthetic proteins.

The amino acid sequence and nucleic acid sequence of annexin-4 is known in the art for different animal species. The nucleic acid sequence encoding human annexin-4 is represented herein by SEQ ID NO: 1. SEQ ID NO:1 encodes the full-length annexin-4 protein from positions 74 to 1039 of SEQ ID NO:1, the amino acid sequence of which is represented herein by SEQ ID NO:2.

In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein. According to the present invention, annexin-4 biological activity can include one or more (or all) of the following biological activities of wild-type annexin-4: phospholipid binding, calcium-dependent phospholipid binding; promotion of membrane fusion; and regulation of or involvement in exocytosis.

Methods of detecting and measuring protein expression and biological activity include, but are not limited to, measurement of transcription of a protein, measurement of translation of a protein, measurement of posttranslational modification of a protein, measurement of the ability of the protein to bind to another protein(s); measurement of the ability of the protein to induce or participate in a particular biological effect. It is noted that an isolated protein of the present invention (including a homologue) is not necessarily required to have the biological activity of the wild-type protein. For example, a protein can be a truncated, mutated or inactive protein, for example. Such proteins are useful in screening assays, for example, or for other purposes such as antibody production. In a preferred embodiment, the isolated proteins of the present invention have a biological activity that is similar to that of the wild-type protein (although not necessarily equivalent).

The present invention includes homologues of annexin-4. As used herein, the term “homologue” is used to refer to a protein or peptide which differs from a naturally occurring protein or peptide (i.e., the “prototype” or “wild-type” protein) by one or more minor modifications or mutations to the naturally occurring protein or peptide, but which maintains the overall basic protein and side chain structure of the naturally occurring form (i.e., such that the homologue is identifiable as being related to the wild-type protein). Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, farnesylation, geranyl geranylation, glycosylation, carboxymethylation, phosphorylation, acetylation, myristoylation, prenylation, palmitation, and/or amidation. A homologue can have either enhanced, decreased, or substantially similar properties as compared to the naturally occurring protein or peptide. Preferred homologues of an annexin-4 protein are described in detail below. It is noted that homologues can include synthetically produced homologues (synthetic peptides or proteins), naturally occurring allelic variants of a given protein, or homologous sequences from organisms other than the organism from which the reference sequence was derived.

Conservative substitutions typically include substitutions within the following groups: glycine and alanine; valine, isoleucine and leucine; aspartic acid, glutamic acid, asparagine, and glutamine; serine and threonine; lysine and arginine; and phenylalanine and tyrosine. Substitutions may also be made on the basis of conserved hydrophobicity or hydrophilicity (Kyte and Doolittle, J. Mol. Biol. (1982) 157: 105-132), or on the basis of the ability to assume similar polypeptide secondary structure (Chou and Fasman, Adv. Enzymol. (1978) 47: 45-148, 1978).

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

Modifications or mutations in protein homologues, as compared to the wild-type protein, either increase, decrease, or do not substantially change, the basic biological activity of the homologue as compared to the naturally occurring (wild-type) protein. With regard to annexin-4, the present invention includes homologues that maintain the basic biological activities of the wild-type protein, as well as homologues that maintain only some of the biological activities of the wild-type protein (e.g., the ability to bind to phospholipids or ability to be bound by a natural antibody, but not exocytosis activity). It is noted that general reference to a homologue having the biological activity of the wild-type protein does not necessarily mean that the homologue has identical biological activity as the wild-type protein, particularly with regard to the level of biological activity. Rather, a homologue can perform the same general biological activity as the wild-type protein, but at a reduced or increased level of activity as compared to the wild-type protein.

In one embodiment of the invention, a homologue of annexin-4 useful in the methods of the invention includes a fragment of the full-length annexin-4. In one embodiment, such a fragment consists essentially of or consists of a fragment of a wild-type annexin-4 protein that is capable of binding to a phospholipid. In another embodiment, such a fragment consists essentially of or consists of a fragment of a wild-type annexin-4 protein that is capable of binding to a natural antibody against annexin-4. In one aspect of the invention, a homologue of annexin-4 comprises, consists essentially of, or consists of, an amino acid sequence that is at least about 50% identical, and more preferably at least about 55% identical, and more preferably at least about 60% identical, and more preferably at least about 65% identical, and more preferably at least about 70% identical, and more preferably at least about 75% identical, and more preferably at least about 80% identical, and even more preferably at least about 85% identical, and even more preferably at least about 90% identical and even more preferably at least about 95% identical, and even more preferably at least about 96% identical, and even more preferably at least about 97% identical, and even more preferably at least about 98% identical, and even more preferably at least about 99% identical (or any percentage between 60% and 99%, in whole single percentage increments) to the natural reference amino acid sequence (e.g., the wild-type annexin-4 protein, such as that represented by SEQ ID NO:2) over a length of the natural sequence that is at least the same as the length of the homologue. A homologue includes a fragment of a natural (full-length or wild-type sequence), including biologically active, partially biologically active (e.g., binds to a ligand or receptor, but may not have further biological activity), biologically inactive, and soluble forms of the natural protein (e.g., if the natural protein is a membrane or insoluble protein).

In one embodiment, an annexin-4 homologue of the present invention comprises, consists essentially of, or consists of an amino acid sequence that is less than 100% identical to the wild-type sequence for annexin-4, or less than about 99% identical, or less than 98% identical, or less than 97% identical, or less than 96% identical, or less than 95% identical, or less than 94% identical, or less than 93% identical, or less than 92% identical, or less than 91% identical, or less than 90% identical to the wild-type annexin-4 sequence (e.g., SEQ ID NO:2), and so on, in increments of whole integers. The isolated annexin-4 homologue of the present invention preferably has at least one biological activity of a naturally occurring or wild-type annexin-4 protein, and most preferably, retains at least one conformational epitope that is bound by a natural antibody against annexin-4 and/or retains the ability to bind to phospholipids.

As used herein, unless otherwise specified, reference to a percent (%) identity refers to an evaluation of homology which is performed using a BLAST homology search. BLAST homology searches can be performed using the BLAST database and software, which offers search programs including: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs, although for the direct comparison of two sequences, BLAST 2 is preferred.

According to the present invention, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

In another embodiment, an isolated protein of the present invention, including an isolated homologue, includes a protein having an amino acid sequence that is sufficiently similar to a naturally occurring protein amino acid sequence that a nucleic acid sequence encoding the homologue is capable of hybridizing under moderate, high, or very high stringency conditions (described below) to (i.e., with) a nucleic acid molecule encoding the naturally occurring protein (i.e., to the complement of the nucleic acid strand encoding the naturally occurring protein amino acid sequence). A “complement” of nucleic acid sequence encoding a protein of the present invention refers to the nucleic acid sequence of the nucleic acid strand that is complementary to the strand which encodes the protein. Methods to deduce a complementary sequence are well known to those skilled in the art.

As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, low stringency hybridization and washing conditions, as referred to herein, refer to conditions which permit isolation of nucleic acid molecules having at least about 50% nucleic acid sequence identity with the nucleic acid molecule being used to probe in the hybridization reaction (i.e., conditions permitting about 50% or less mismatch of nucleotides). Moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids.

In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

The present invention also includes a fusion protein or a chimeric protein that includes a desired protein-containing domain (e.g., annexin-4 or a homologue or fragment thereof) attached to one or more fusion segments or additional proteins or peptides. Suitable fusion segments for use with the present invention include, but are not limited to, segments that can: enhance a protein's stability; provide other desirable biological activity; and/or assist with the purification of a protein (e.g., by affinity chromatography), or provide another protein function (e.g., as in a chimeric protein). A suitable fusion segment can be a domain of any size that has the desired function (e.g., imparts increased stability, solubility, action or biological activity; simplifies purification of a protein; or provides the additional protein function). Fusion segments can be joined to amino and/or carboxyl termini of the domain of the desired protein and can be susceptible to cleavage in order to enable straight-forward recovery of the protein. In one embodiment a suitable fusion segment or protein with which a chimeric or fusion protein can be produced is an antibody fragment and particularly, the Fc portion of an immunoglobulin protein. Any fusion or chimera partner that enhances the stability or half-life of annexin-4 in vivo, for example, is contemplated for use in the present invention.

In one embodiment of the present invention, any of the above-described amino acid sequences, as well as homologues of such sequences, can be produced with from at least one, and up to about 20, additional heterologous amino acids flanking each of the C- and/or N-terminal end of the given amino acid sequence. The resulting protein or polypeptide can be referred to as “consisting essentially of” a given amino acid sequence. According to the present invention, the heterologous amino acids are a sequence of amino acids that are not naturally found (i.e., not found in nature, in vivo) flanking the given amino acid sequence or which would not be encoded by the nucleotides that flank the naturally occurring nucleic acid sequence encoding the given amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a given amino acid sequence that can be flanked by from at least one, and up to as many as about 60, additional heterologous nucleotides at each of the 5′ and/or the 3′ end of the nucleic acid sequence encoding the given amino acid sequence. The heterologous nucleotides are not naturally found (i.e., not found in nature, in vivo) flanking the nucleic acid sequence encoding the given amino acid sequence as it occurs in the natural gene.

According to the present invention, the minimum size of a protein, portion of a protein (e.g. a fragment, portion, domain, etc.), or region or epitope of a protein, is a size sufficient to serve as an epitope or conserved binding surface for the generation of an antibody or as a target in an in vitro assay, or to bind to a phospholipid. In one embodiment, a protein of the present invention is at least about 4, 5, 6, 7 or 8 amino acids in length (e.g., suitable for an antibody epitope or as a detectable peptide in an assay), or at least about 25 amino acids in length, or at least about 50 amino acids in length, or at least about 100 amino acids in length, or at least about 150 amino acids in length, and so on, in any length between 4 amino acids and up to the full length of a protein or portion thereof or longer, in whole integers (e.g., 8, 9, 10, . . . 25, 26, . . . ).

One embodiment of the present invention relates to an isolated nucleic acid molecule comprising, consisting essentially of, or consisting of a nucleic acid sequence that encodes any of the annexin-4 proteins described herein, including a homologue or fragment of any of such proteins, as well as nucleic acid sequences that are fully complementary thereto. In accordance with the present invention, an isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene. An isolated nucleic acid molecule that includes a gene is not a fragment of a chromosome that includes such gene, but rather includes the coding region and regulatory regions associated with the gene, but no additional genes that are naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein or domain of a protein.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on the biological activity of the protein as described herein. Protein homologues (e.g., proteins encoded by nucleic acid homologues) have been discussed in detail above.

A nucleic acid molecule homologue can be produced using a number of methods known to those skilled in the art (see, for example, Sambrook et al., Molecular Cloning: A Laboratory Manual, Cold Spring Harbor Labs Press, 1989). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene.

The minimum size of a nucleic acid molecule of the present invention is a size sufficient to encode a protein having the desired biological activity or a protein that comprises at least one conformational epitope that is bound by a natural antibody against the protein, or is sufficient to form a probe or oligonucleotide primer that is capable of forming a stable hybrid with the complementary sequence of a nucleic acid molecule encoding the natural protein (e.g., under moderate, high or very high stringency conditions). As such, the size of the nucleic acid molecule encoding such a protein can be dependent on nucleic acid composition and percent homology or identity between the nucleic acid molecule and complementary sequence as well as upon hybridization conditions per se (e.g., temperature, salt concentration, and formamide concentration). The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a portion of a protein-encoding sequence or a nucleic acid sequence encoding a full-length protein.

One embodiment of the present invention is a recombinant nucleic acid molecule comprising an isolated nucleic acid molecule of the present invention. According to the present invention, a recombinant nucleic acid molecule includes at least one isolated nucleic acid molecule of the present invention that is linked to a heterologous nucleic acid sequence. Such a heterologous nucleic acid sequence is typically a recombinant nucleic acid vector (e.g., a recombinant vector) which is suitable for cloning, sequencing, and/or otherwise manipulating the nucleic acid molecule, such as by expressing and/or delivering the nucleic acid molecule into a host cell to form a recombinant cell. Such a vector contains heterologous nucleic acid sequences, that is nucleic acid sequences that are not naturally found adjacent to nucleic acid molecules of the present invention, although the vector can also contain regulatory nucleic acid sequences (e.g., promoters, untranslated regions) which are naturally found adjacent to nucleic acid molecules of the present invention (discussed in detail below). The vector can be either RNA or DNA, either prokaryotic or eukaryotic, and typically is a virus or a plasmid. The vector can be maintained as an extrachromosomal element (e.g., a plasmid) or it can be integrated into the chromosome. The entire vector can remain in place within a host cell, or under certain conditions, the plasmid DNA can be deleted, leaving behind the nucleic acid molecule of the present invention. The integrated nucleic acid molecule can be under chromosomal promoter control, under native or plasmid promoter control, or under a combination of several promoter controls. Single or multiple copies of the nucleic acid molecule can be integrated into the chromosome. As used herein, the phrase “recombinant nucleic acid molecule” is used primarily to refer to a recombinant vector into which has been ligated the nucleic acid sequence to be cloned, manipulated, transformed into the host cell (i.e., the insert).

Another embodiment of the present invention relates to an antibody that selectively binds to a protein, such as annexin-4 or to a phospholipid (e.g., phosphotidylcholine or phosphoglycerol). As used herein, the term “selectively binds to” refers to the specific binding of one protein to another protein, to a lipid, or to a carbohydrate moiety (e.g., the binding of an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

According to the present invention, an “epitope” of a given protein or peptide or other molecule is generally defined, with regard to antibodies, as a part of or site on a larger molecule to which an antibody or antigen-binding fragment thereof will bind, and against which an antibody will be produced. The term epitope can be used interchangeably with the term “antigenic determinant”, “antibody binding site”, or “conserved binding surface” of a given protein or antigen. More specifically, an epitope can be defined by both the amino acid residues involved in antibody binding and also by their conformation in three dimensional space (e.g., a conformational epitope or the conserved binding surface). An epitope can be included in peptides as small as about 4-6 amino acid residues, or can be included in larger segments of a protein, and need not be comprised of contiguous amino acid residues when referring to a three dimensional structure of an epitope, particularly with regard to an antibody-binding epitope. Antibody-binding epitopes are frequently conformational epitopes rather than a sequential epitope (i.e., linear epitope), or in other words, an epitope defined by amino acid residues arrayed in three dimensions on the surface of a protein or polypeptide to which an antibody binds. As mentioned above, the conformational epitope is not comprised of a contiguous sequence of amino acid residues, but instead, the residues are perhaps widely separated in the primary protein sequence, and are brought together to form a binding surface by the way the protein folds in its native conformation in three dimensions.

One of skill in the art can identify and/or assemble conformational epitopes and/or sequential epitopes using known techniques, including mutational analysis (e.g., site-directed mutagenesis); protection from proteolytic degradation (protein footprinting); mimotope analysis using, e.g., synthetic peptides and pepscan, BIACORE or ELISA; antibody competition mapping; combinatorial peptide library screening; matrix-assisted laser desorption/ionization time-of-flight (MALDI-TOF) mass spectrometry; or three-dimensional modeling (e.g., using any suitable software program, including, but not limited to, MOLSCRIPT 2.0 (Avatar Software AB, Heleneborgsgatan 21C, SE-11731 Stockholm, Sweden), the graphical display program O (Jones et. al., Acta Crystallography, vol. A47, p. 110, 1991), the graphical display program GRASP, or the graphical display program INSIGHT). For example, one model the three-dimensional structure of annexin-4 and predict the conformational epitope of antibody binding to this structure. Indeed, one can use one or any combination of such techniques to define the antibody binding epitope.

According to the present invention, antibodies are characterized in that they comprise immunoglobulin domains and as such, they are members of the immunoglobulin superfamily of proteins. Generally speaking, an antibody molecule comprises two types of chains. One type of chain is referred to as the heavy or H chain and the other is referred to as the light or L chain. The two chains are present in an equimolar ratio, with each antibody molecule typically having two H chains and two L chains. The two H chains are linked together by disulfide bonds and each H chain is linked to a L chain by a disulfide bond. There are only two types of L chains referred to as lambda (λ) and kappa (κ) chains. In contrast, there are five major H chain classes referred to as isotypes. The five classes include immunoglobulin M (IgM or μ), immunoglobulin D (IgD or δ), immunoglobulin G (IgG or λ), immunoglobulin A (IgA or α), and immunoglobulin E (IgE or ε). The distinctive characteristics between such isotypes are defined by the constant domain of the immunoglobulin and are discussed in detail below. Human immunoglobulin molecules comprise nine isotypes, IgM, IgD, IgE, four subclasses of IgG including IgG1 (γ1), IgG2 (γ2), IgG3 (γ3) and IgG4 (γ4), and two subclasses of IgA including IgA1 (α1) and IgA2 (α2). In humans, IgG subclass 3 and IgM are the most potent complement activators (classical complement system), while IgG subclass 1 and to an even lesser extent, 2, are moderate to low activators of the classical complement system. IgG4 subclass does not activate the complement system (classical or alternative). The only human immunoglobulin isotype known to activate the alternative complement system is IgA. In mice, the IgG subclasses are IgG1, IgG2a, IgG2b and IgG3. Murine IgG1 does not activate complement, while IgG2a, IgG2b and IgG3 are complement activators.

Each H or L chain of an immunoglobulin molecule comprises two regions referred to as L chain variable domains (V_{L} domains) and L chain constant domains (C_{L} domains), and H chain variable domains (V_{H} domains) and H chain constant domains (C_{H} domains). A complete C_{H} domain comprises three sub-domains (CH1, CH2, CH3) and a hinge region. Together, one H chain and one L chain can form an arm of an immunoglobulin molecule having an immunoglobulin variable region. A complete immunoglobulin molecule comprises two associated (e.g., di-sulfide linked) arms. Thus, each arm of a whole immunoglobulin comprises a V_{H+L} region, and a C_{H+L} region. As used herein, the term “variable region” or “V region” refers to a V_{H+L} region (also known as an Fv fragment), a V_{L} region or a V_{H} region. Also as used herein, the term “constant region” or “C region” refers to a C_{H+L} region, a C_{L} region or a C_{H} region.

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

The C_{H} domain defines the isotype of an immunoglobulin and confers different functional characteristics depending upon the isotype. For example, μ constant regions enable the formation of pentameric aggregates of IgM molecules and α constant regions enable the formation of dimers.

The antigen specificity of an immunoglobulin molecule is conferred by the amino acid sequence of a variable, or V, region. As such, V regions of different immunoglobulin molecules can vary significantly depending upon their antigen specificity. Certain portions of a V region are more conserved than others and are referred to as framework regions (FW regions). In contrast, certain portions of a V region are highly variable and are designated hypervariable regions. When the V_{L} and V_{H} domains pair in an immunoglobulin molecule, the hypervariable regions from each domain associate and create hypervariable loops that form the antigen binding sites (antigen combining sites). Thus, the hypervariable loops determine the specificity of an immunoglobulin and are termed complementarity-determining regions (CDRs) because their surfaces are complementary to antigens.

Further variability of V regions is conferred by combinatorial variability of gene segments that encode an immunoglobulin V region. Immunoglobulin genes comprise multiple germline gene segments which somatically rearrange to form a rearranged immunoglobulin gene that encodes an immunoglobulin molecule. V_{L} regions are encoded by a L chain V gene segment and J gene segment (joining segment). V_{H} regions are encoded by a H chain V gene segment, D gene segment (diversity segment) and J gene segment (joining segment).

Both a L chain and H chain V gene segment contain three regions of substantial amino acid sequence variability. Such regions are referred to as L chain CDR1, CDR2 and CDR3, and H chain CDR1, CDR2 and CDR3, respectively. The length of an L chain CDR1 can vary substantially between different V_{L} regions. For example, the length of CDR1 can vary from about 7 amino acids to about 17 amino acids. In contrast, the lengths of L chain CDR2 and CDR3 typically do not vary between different V_{L} regions. The length of a H chain CDR3 can vary substantially between different V_{H} regions. For example, the length of CDR3 can vary from about 1 amino acid to about 20 amino acids. Each H and L chain CDR region is flanked by FW regions.

Other functional aspects of an immunoglobulin molecule include the valency of an immunoglobulin molecule, the affinity of an immunoglobulin molecule, and the avidity of an immunoglobulin molecule. As used herein, affinity refers to the strength with which an immunoglobulin molecule binds to an antigen at a single site on an immunoglobulin molecule (i.e., a monovalent Fab fragment binding to a monovalent antigen). Affinity differs from avidity which refers to the sum total of the strength with which an immunoglobulin binds to an antigen. Immunoglobulin binding affinity can be measured using techniques standard in the art, such as competitive binding techniques, equilibrium dialysis or BIAcore methods. As used herein, valency refers to the number of different antigen binding sites per immunoglobulin molecule (i.e., the number of antigen binding sites per antibody molecule of antigen binding fragment). For example, a monovalent immunoglobulin molecule can only bind to one antigen at one time, whereas a bivalent immunoglobulin molecule can bind to two or more antigens at one time, and so forth.

In one embodiment, an antibody useful in the invention is a bi- or multi-specific antibody. A bi-specific (or multi-specific) antibody is capable of binding two (or more) antigens, as with a divalent (or multivalent) antibody, but in this case, the antigens are different antigens (i.e., the antibody exhibits dual or greater specificity).

In one embodiment, antibodies of the present invention include humanized antibodies. Humanized antibodies are molecules having an antigen binding site derived from an immunoglobulin from a non-human species, the remaining immunoglobulin-derived parts of the molecule being derived from a human immunoglobulin. The antigen binding site may comprise either complete variable regions fused onto human constant domains or only the complementarity determining regions (CDRs) grafted onto appropriate human framework regions in the variable domains. Humanized antibodies can be produced, for example, by modeling the antibody variable domains, and producing the antibodies using genetic engineering techniques, such as CDR grafting (described below). A description various techniques for the production of humanized antibodies is found, for example, in Morrison et al. (1984) Proc. Natl. Acad. Sci. USA 81:6851-55; Whittle et al. (1987) Prot. Eng. 1:499-505; Co et al. (1990) J. Immunol. 148:1149-1154; Co et al. (1992) Proc. Natl. Acad. Sci. USA 88:2869-2873; Carter et al. (1992) Proc. Natl. Acad. Sci. 89:4285-4289; Routledge et al. (1991) Eur. J. Immunol. 21:2717-2725 and PCT Patent Publication Nos. WO 91/09967; WO 91/09968 and WO 92/113831.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies, humanized antibodies (discussed above), antibodies that can bind to more than one epitope (e.g., bi-specific antibodies), or antibodies that can bind to one or more different antigens (e.g., bi- or multi-specific antibodies), may also be employed in the invention.

Genetically engineered antibodies of the invention include those produced by standard recombinant DNA techniques involving the manipulation and re-expression of DNA encoding antibody variable and/or constant regions. Particular examples include, chimeric antibodies, where the V_{H} and/or V_{L} domains of the antibody come from a different source as compared to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

The invention also extends to the use of non-antibody polypeptides, sometimes referred to as antigen binding partners or antigen binding polypeptides, that have been designed to bind selectively to a protein according to the present invention. Examples of the design of such polypeptides, which possess a prescribed ligand specificity are given in Beste et al. (Proc. Natl. Acad. Sci. 96:1898-1903, 1999), incorporated herein by reference in its entirety.

Accordingly, one embodiment of the present invention includes the use of an antibody, antigen binding fragment thereof, or antigen-binding polypeptide that is a competitive inhibitor of the binding of annexin-4 or a phospholipid, and particularly phosphatidylcholine or phosphoglycerol, to a natural antibody that binds to annexin-4 or the phospholipid, or, in the case of annexin-4, that is a competitive inhibitor of a pathogenic monoclonal antibody against annexin-4 (e.g., monoclonal antibody B4 described herein), or in the case of phospholipids, that is a competitive inhibitor of a pathogenic monoclonal antibody against phospholipids (e.g., monoclonal antibody C2 described herein). Such antibodies and related binding polypeptides are useful for inhibiting physiological damage and effects associated with ischemic-reperfusion injury.

According to the present invention, a competitive inhibitor of the binding of a protein (annexin-4) or lipid (e.g., phosphatidylcholine or phosphoglycerol) to an antibody (e.g., a natural antibody or a pathogenic antibody described herein) is an inhibitor (e.g., another antibody or antigen binding fragment or a polypeptide) that binds to annexin-4 or the lipid at the same or similar epitope as the natural or pathogenic antibody, such that binding of the natural or pathogenic antibody to its antigen (the protein or lipid) is inhibited. A competitive inhibitor may, in one embodiment, bind to the target (e.g., annexin-4) with a greater affinity for the target than the natural or pathogenic antibody. A competitive inhibitor is preferably used herein to inhibit the binding of natural antibodies to annexin-4 or to the phospholipids phosphatidylcholine or phosphoglycerol to inhibit or prevent the physiological damage or effects caused by ischemia-reperfusion injury.

For example, one embodiment of the invention relates to the use of an isolated antibody, antigen binding fragment thereof, or antigen-binding polypeptide, that specifically binds to annexin-4, wherein the antibody or fragment thereof or polypeptide competitively inhibits mAb B4, or another pathogenic antibody that binds to annexin-4, for specific binding to annexin-4, and wherein, when the antibody or fragment thereof or polypeptide binds to annexin-4, the ability of the mAb B4 or similar pathogenic antibody to bind to annexin-4 and/or to initiate ischemia-reperfusion injury, is inhibited or prevented.

Another embodiment of the invention relates to the use of an isolated antibody, antigen binding fragment thereof, or antigen-binding polypeptide, that specifically binds to a phospholipid including phosphatidylcholine or phosphoglycerol, wherein the antibody or fragment or polypeptide competitively inhibits mAb C2, or another pathogenic antibody that binds to such phospholipids, for specific binding to such phospholipids, and wherein, when the antibody or fragment thereof or polypeptide binds to such phospholipids, the ability of the mAb C2 or similar pathogenic antibody to bind to such phospholipids and/or to initiate ischemia-reperfusion injury, is inhibited or prevented.

Another embodiment of the invention relates to the use of an isolated antibody or antigen binding fragment thereof or antigen-binding polypeptide that specifically binds to phosphatidylcholine or phosphoglycerol, wherein the antibody or fragment thereof or polypeptide competitively inhibits the specific binding of natural antibodies in an individual to phosphatidylcholine or phosphoglycerol, and wherein, when the antibody or fragment thereof or polypeptide binds to phosphatidylcholine or phosphoglycerol, the ability of natural antibodies to bind to phosphatidylcholine or phosphoglycerol and/or to initiate ischemia-reperfusion injury, is inhibited or prevented.

Another embodiment of the invention relates to the use of an isolated antibody or antigen binding fragment thereof or antigen-binding polypeptide that specifically binds to annexin-4, wherein the antibody or fragment thereof or polypeptide competitively inhibits the specific binding of natural antibodies in an individual to annexin-4, and wherein, when the antibody or fragment thereof or polypeptide binds to annexin-4, the ability of natural antibodies to bind to annexin-4 and/or to initiate ischemia-reperfusion injury, is inhibited or prevented.

Competition assays can be performed using standard techniques in the art (e.g., competitive ELISA or other binding assays). For example, competitive inhibitors can be detected and quantitated by their ability to inhibit the binding of an antigen to a known, labeled antibody (e.g., the mAb B4) or to sera or another composition that is known to contain antibodies against the particular antigen (e.g., sera known to contain natural antibodies against the antigen).

The present invention also includes the use of other inhibitors, including small molecules, that are designed or selected to be inhibitors of the binding of natural antibodies to phospholipids (e.g., phosphatidylcholine or phosphoglycerol) or annexin-4. Such agents include, for example, compounds that are products of rational drug design, natural products, and compounds having partially defined regulatory properties. Such a molecule can be a protein-based compound, a carbohydrate-based compound, a lipid-based compound, a nucleic acid-based compound, a natural organic compound, or a synthetically derived organic compound. Such an agent can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the same building blocks) or by rational drug design. See for example, Maulik et al., 1997, Molecular Biotechnology: Therapeutic Applications and Strategies, Wiley-Liss, Inc., which is incorporated herein by reference in its entirety.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands against a desired target, and then optimize the lead molecules by either random or directed design strategies. Methods of molecular diversity are described in detail in Maulik, et al., supra.

In a rational drug design procedure, the three-dimensional structure of a regulatory compound can be analyzed by, for example, nuclear magnetic resonance (NMR) or X-ray crystallography. This three-dimensional structure can then be used to predict structures of potential compounds, such as potential regulatory agents by, for example, computer modeling. The predicted compound structure can be used to optimize lead compounds derived, for example, by molecular diversity methods. In addition, the predicted compound structure can be produced by, for example, chemical synthesis, recombinant DNA technology, or by isolating a mimetope from a natural source (e.g., plants, animals, bacteria and fungi).

Various other methods of structure-based drug design are disclosed in Maulik et al., 1997, supra. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

The invention also includes any agent described herein, including liposomes, lipid moieties, antibodies, antigen-binding fragments, antigen-binding polypeptides, proteins, small molecules, nucleic acids (e.g., apatmers) and similar agents that are used to target therapeutic moieties to a site for the prevention or inhibition of ischemic-reperfusion injury. For example, one can use the information provided by the identification of the phospholipids and annexin-4 targets described herein, as well as the identification of the pathogenic antibodies described herein, to target any therapeutic modality to the site of injury in ischemia-reperfusion injury. For example, the antigen-combining sites of any of the pathogenic antibodies described herein (e.g., B4 or C2, or any pathogenic antibody that binds to annexin-4 or phospholipids, pathogenicity being defined by the ability to transfer any measure of ischemia-reperfusion injury to an immune competent host), can be used to provide and develop novel targeting agents. In one embodiment, the invention provides an agent that can include, but is not limited to, an antibody or an antigen-binding fragment thereof, or a polypeptide or other molecule, wherein the agent binds to the same or similar site on annexin-4 or a phospholipid as any of the pathogenic antibodies described herein, or that makes use of the antigen-combining sites of any of the antibodies described herein, to target any therapeutic moiety (e.g., a drug) useful in treating any aspect of ischemia-reperfusion injury, or a disease or condition in which ischemia-reperfusion injury occurs, to a specific site in an individual. The specific site will accordingly be a site of injury or of possible injury due to ischemia-reperfusion. Such targeting agents (targeting moieties) can be designed or identified using the primary or tertiary structure of pathogenic antibodies described herein (e.g., by computer design or other structure analysis), by competitive assays (e.g., competition with pathogenic antibodies such as those described herein), by epitope mapping of the antigens and design of binding partners that bind to the conformational epitope, and simply by producing non-pathogenic variants and fragments of any pathogenic antibody or polypeptide described herein. Other methods of producing such targeting agents will be known to those of skill in the art.

Targeting agents can be linked to (or designed to be) any suitable agent for the treatment or prevention of ischemia reperfusion injury or a disease or condition associated therewith. In one aspect, the targeting agents are linked to or associated with any of the therapeutic modalities described previously herein, such as liposomes or lipid moieties, annexin-4 or homologues or fragments thereof, or other proteins and agents described above.

The present invention also includes compositions that comprise any of the liposomes or other lipid moieties, antibodies, proteins, and other agents useful in the present invention. In one embodiment, a composition includes an agent useful in the present invention (e.g., liposomes, annexin-4 protein, or another agent) and one or more pharmaceutically acceptable carriers. According to the present invention, a “pharmaceutically acceptable carrier” includes pharmaceutically acceptable excipients and/or pharmaceutically acceptable delivery vehicles, which are suitable for use in the administration of a formulation or composition to a suitable in vivo site. A suitable in vivo site is preferably any site wherein ischemia-reperfusion injury is occurring or is expected to occur. Preferred pharmaceutically acceptable carriers are capable of maintaining an agent used in a formulation of the invention in a form that, upon arrival of the agent at the target site in a patient, the agent is capable of acting on its target (e.g., a cell or tissue that is showing signs of cellular stress or symptoms of ischemia-reperfusion injury), preferably resulting in a therapeutic benefit to the patient. A delivery vehicle for a protein or agent can include a liposome, although in preferred embodiments of the invention, the liposome is preferably also a therapeutic agent as described herein (e.g., the liposome can serve one or both functions).

Suitable excipients of the present invention include excipients or formularies that transport or help transport, but do not specifically target a composition to a cell or tissue (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Formulations of the present invention can be sterilized by conventional methods and/or lyophilized.

One type of pharmaceutically acceptable carrier includes a controlled release formulation that is capable of slowly releasing a composition of the present invention into an individual. As used herein, a controlled release formulation comprises an agent of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other suitable carriers include any carrier that can be bound to or incorporated with the agent that extends that half-life of the agent to be delivered. Such a carrier can include any suitable protein carrier or even a fusion segment that extends the half-life of a protein when delivered in vivo. Suitable delivery vehicles have been previously described herein, and include, but are not limited to liposomes, viral vectors or other delivery vehicles, including ribozymes. Natural lipid-containing delivery vehicles include cells and cellular membranes. Artificial lipid-containing delivery vehicles include liposomes and micelles. A delivery vehicle of the present invention can be modified to target to a particular site in a patient, thereby targeting and making use of an inhibitory agent at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

A pharmaceutically acceptable carrier which is capable of targeting is referred to as a “targeting delivery vehicle.” Targeting delivery vehicles of the present invention are capable of delivering a formulation, including an inhibitory agent, to a target site in a patient. A “target site” refers to a site in a patient to which one desires to deliver a therapeutic formulation. For example, a target site can be any cell or tissue which is targeted by direct injection or delivery using liposomes or other delivery vehicles. A delivery vehicle of the present invention can be modified to target to a particular site in an individual, thereby targeting and making use of the agent at that site. Suitable modifications include manipulating the chemical formula of the lipid portion of the delivery vehicle and/or introducing into the vehicle a compound capable of specifically targeting a delivery vehicle to a preferred site, for example, a preferred cell or tissue type. Specifically, targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands.

One embodiment of the present invention relates to the use of any of the agents described herein, including combinations thereof, to treat or prevent ischemia-reperfusion injury. As discussed above, the present invention can be used to treat or prevent any ischemia-reperfusion injury that occurs in any organ or tissue, including, but not limited to, intestinal ischemia-reperfusion injury, renal ischemia-reperfusion injury, cardiac ischemia-reperfusion injury, ischemia-reperfusion injury of other internal organs such as the lung or liver, central nervous system ischemia-reperfusion injury, ischemia-reperfusion injury of the limbs or digits, or ischemia-reperfusion injury of any transplanted organ or tissue. Also as noted above, ischemia-reperfusion injury can occur in conjunction with a variety of conditions including, but not limited to, stroke, traumatic brain injury, spinal cord injury, trauma-induced hypovolemic shock, and autoimmune and inflammatory diseases such as rheumatoid arthritis (e.g., which can be greatly worsened by ischemic injury of the synovium). Other conditions and diseases in which ischemia-reperfusion injury occurs will be known to those of skill in the art.

Another embodiment of the present invention relates to the use of any of the agents described herein, including combinations thereof, to treat autoimmune disease. As discussed above, autoimmune diseases can include, but are not limited to, rheumatoid arthritis, systemic lupus erythematosus, multiple sclerosis, myasthenia gravis, insulin-dependent diabetes mellitus, acute disseminated encephalomyelitis, Addison's disease, antiphospholipid antibody syndrome, autoimmune hepatitis, Crohn's disease, Goodpasture's syndrome, Graves' disease, Guillain-Barré syndrome, Hashimoto's disease, idiopathic thrombocytopenic purpura, pemphigus, Sjögren's syndrome, and Takayasu's arteritis. In particular, the method of the invention is useful for the prevention and/or inhibition of reperfusion injury associated with such diseases, for example, reperfusion injury due to chronic or intermittent ischemic events.

Another embodiment of the present invention relates to the use of any of the agents described herein, including combinations thereof, to treat an inflammatory disease, including inflammation due to infection by a pathogen. In particular, the method of the invention is useful for the prevention and/or inhibition of reperfusion injury associated with such diseases, for example, reperfusion injury due to chronic or intermittent ischemic events.

The methods of the invention includes administering to an individual that has, or is at risk of experiencing or developing, ischemia-reperfusion injury (or a disease or condition associated with ischemia-reperfusion injury), at least one agent that blocks or inhibits the interaction of natural antibodies in an individual with: (1) annexin-4 expressed on the surface of a cell that is in or adjacent to a tissue that is undergoing ischemia-reperfusion injury (or is at risk of undergoing ischemia-reperfusion injury); and/or (2) a phospholipid expressed on the surface of a cell that is in or adjacent to a tissue that is undergoing ischemia-reperfusion injury (or is at risk of undergoing ischemia-reperfusion injury). Particularly preferred agents have been described in detail above and include, but are not limited to, (1) a liposome or lipid moiety comprising phospholipids (e.g., phosphatidylcholine, phosphoglycerol, lysophosphatidylcholine, phosphatidic acid, phosphatidylethanolamine, and/or phosphatidylserine, and/or derivatives of any of such phospholipids); (2) an isolated annexin-4 protein or biologically active homologue thereof; and/or (3) an annexin-4-liposome (or other lipid) complex, wherein the liposome/lipid portion of the complex comprises the phospholipids as described in (1) above. In one embodiment, the phospholipids contained in the liposomes or lipid moieties described herein consist essentially of or consist of the phospholipids selected from phosphatidylcholine, phosphoglycerol, lysophosphatidylcholine, phosphatidic acid, phosphatidylethanolamine, and/or phosphatidylserine, and/or derivatives of any of such phospholipids, with the phospholipids, phosphatidylcholine and/or phosphoglycerol being one preferred embodiment. In one embodiment, any of the above-described liposomes or lipid moieties further comprise cholesterol or any other lipid or lipid derivative that is useful for stabilizing the bilayer of lipids in a liposome or lipid moieties and/or decreasing leakage of encapsulated material. In one embodiment, any of the above-described liposomes or lipid moieties further comprise antioxidants such as α-tocopherol or β-hydroxytoluidine. Such antioxidants are useful for inhibiting oxidation of the lipids in liposomes. Other suitable agents for use in the invention have also been described above and are contemplated for use in this method of the invention.

It is noted that this embodiment of the present invention is specifically directed to the treatment or prevention of ischemia-reperfusion injury, and as such, it is not required that the related condition or causative factor that initially caused or may cause the ischemia-reperfusion injury be significantly reduced or “cured”. The method of the present invention is fully effective to prevent or reduce damage or injury associated with ischemia-reperfusion or to improve or reduce at least one symptom of such injury. Therefore, administration of an agent or formulation described herein is useful for the prevention or inhibition of ischemia-reperfusion injury, although it is not required that all such injury be completely prevented, but it is preferred that the patient experience at least one therapeutic benefit from the use of the agent or formulation. When the compositions and agents of the invention are used more generally to treat a disease or condition described herein in which reperfusion injury can cause damage (e.g., autoimmune disease or inflammation), the methods of the invention are also not required to cure or substantially eliminate or reduce all symptoms of the disease or condition, although the individual may achieve substantial therapeutic benefit from the treatment. Preferably, damage to cells, tissues, and/or organs due to reperfusion injury or the presence of the mechanisms described herein (e.g., characterized by annexin-4 or phospholipid targeting by the disease) is prevented or reduced in an individual receiving the treatment.

In accordance with the present invention, determination of acceptable protocols to administer an agent, composition or formulation, including the route of administration and the effective amount of an agent to be administered to an individual, can be accomplished by those skilled in the art. An agent of the present invention can be administered in vivo or ex vivo. Suitable in vivo routes of administration can include, but are not limited to, oral, nasal, inhaled, topical, intratracheal, transdermal, rectal, intestinal, intra-luminal, and parenteral routes. Preferred parenteral routes can include, but are not limited to, subcutaneous, intradermal, intravenous, intramuscular, intraarterial, intrathecal and intraperitoneal routes. Preferred topical routes include inhalation by aerosol (i.e., spraying) or topical surface administration to the skin of an animal. Preferably, an agent is administered by nasal, inhaled, intratracheal, topical, or systemic routes (e.g., intraperitoneal, intravenous). Ex vivo refers to performing part of the administration step outside of the patient. Preferred routes of administration for antibodies include parenteral routes and aerosol/nasal/inhaled routes.

Intravenous, intraperitoneal, and intramuscular administrations can be performed using methods standard in the art. Aerosol (inhalation) delivery can be performed using methods standard in the art (see, for example, Stribling et al., Proc. Natl. Acad. Sci. USA 189:11277-11281, 1992, which is incorporated herein by reference in its entirety). Carriers suitable for aerosol delivery are described above. Devices for delivery of aerosolized formulations include, but are not limited to, pressurized metered dose inhalers (MDI), dry powder inhalers (DPI), and metered solution devices (MSI), and include devices that are nebulizers and inhalers. Oral delivery can be performed by complexing a therapeutic composition of the present invention to a carrier capable of withstanding degradation by digestive enzymes in the gut of an individual. Examples of such carriers, include plastic capsules or tablets, such as those known in the art. Administration of a composition locally within the area of a target cell refers to injecting the composition centimeters and preferably, millimeters from the target cell or tissue.

In humans, it known in the art that, using conventional methods for aerosol delivery, only about 10% of the delivered solution typically enters the deep airways, even using an inhaler. If the aerosolized delivery is by direct inhalation, one may assume a dosage of about 10% of that administered by nebulization methods. Finally, one of skill in the art will readily be capable of converting an animal dosage to a human dosage using alometric scaling. For example, essentially, a scale of dosage from mouse to human is based on the clearance ratio of a compound and the body surface of the mouse. The conversion for mg/kg is 1/12th of the “no observed adverse event level” (NOEL) to obtain the concentration for human dosage. This calculation assumes that the elimination between mouse and human is the same, which is believed to be the case for antibodies, for example.

A preferred single dose of an agent, including proteins, small molecules and antibodies, for use in any method described herein, comprises between about 0.01 microgram×kilogram⁻¹ and about 10 milligram×kilogram⁻¹ body weight of an individual. A more preferred single dose of an agent comprises between about 1 microgram×kilogram⁻¹ and about 10 milligram×kilogram⁻¹ body weight of an individual. An even more preferred single dose of an agent comprises between about 5 microgram×kilogram⁻¹ and about 7 milligram×kilogram⁻¹ body weight of an individual. An even more preferred single dose of an agent comprises between about 10 microgram×kilogram⁻¹ and about 5 milligram×kilogram⁻¹ body weight of an individual. A particularly preferred single dose of an agent comprises between about 0.1 milligram×kilogram⁻¹ and about 5 milligram×kilogram⁻¹ body weight of an animal, if the an agent is delivered by aerosol. Another particularly preferred single dose of an agent comprises between about 0.1 microgram×kilogram⁻¹ and about 10 microgram×kilogram⁻¹ body weight of an individual, if the agent is delivered parenterally.

In one embodiment, an appropriate single dose of a protein:liposome complex of the present invention is from about 0.1 μg to about 100 μg per kg body weight of the patient to which the complex is being administered. In another embodiment, an appropriate single dose is from about 1 μg to about 10 μg per kg body weight. In another embodiment, an appropriate single dose of protein:lipid complex is at least about 0.1 μg of protein:lipid complex, more preferably at least about 1 μg of protein:lipid complex, even more preferably at least about 10 μg of protein:lipid complex, even more preferably at least about 50 μg of protein:lipid complex, and even more preferably at least about 100 μg of protein:lipid complex.

A preferred single dose of an antibody comprises between about 1 ng×kilogram⁻¹ and about less than 1 mg×kilogram⁻¹ body weight of an individual. A more preferred single dose of an antibody comprises between about 20 ng×kilogram⁻¹ and about 600 μg×kilogram⁻¹ body weight of the individual. An even more preferred single dose of an antibody, particularly when the antibody formulation is delivered by nebulization, comprises between about 20 ng×kilogram⁻¹ and about 600 μg×kilogram⁻¹ body weight of the individual, and more preferably, between about 20 ng×kilogram⁻¹ and about 500 μg×kilogram⁻¹, and more preferably, between about 20 ng×kilogram⁻¹ and about 400 μg×kilogram⁻¹, and more preferably, between about 20 ng×kilogram⁻¹ and about 300 μg×kilogram⁻¹, and more preferably, between about 20 ng×kilogram⁻¹ and about 200 μg×kilogram⁻¹, and more preferably, between about 20 ng×kilogram⁻¹ and about 100 μg×kilogram⁻¹, and more preferably, between about 20 ng×kilogram⁻¹ and about 50 μg×kilogram⁻¹ body weight of the individual.

In another embodiment, the protein or antibody is administered at a dose of less than about 500 μg antibody per milliliter of formulation, and preferably, less than about 250 μg protein or antibody per milliliter of formulation, and more preferably, less than about 100 μg protein or antibody per milliliter of formulation, and more preferably, less than about 50 μg protein or antibody per milliliter of formulation, and more preferably, less than about 40 μg protein or antibody per milliliter of formulation, and more preferably, less than about 30 μg protein or antibody per milliliter of formulation, and more preferably, less than about 20 μg protein or antibody per milliliter of formulation, and more preferably, less than about 10 μg protein or antibody per milliliter of formulation, and even more preferably, between about 5 μg protein or antibody and about 10 μg protein or antibody per milliliter of formulation.

With particular regard to the method of inhibiting or preventing ischemia-reperfusion injury, an effective amount of an agent, and particularly a liposome, protein, antibody, drug or combination thereof, to administer to an individual is an amount that measurably inhibits (or prevents) histological damage, including oxidative damage or cell death, in the individual as compared to in the absence of administration of the agent. A suitable single dose of an inhibitory agent to administer to an individual is a dose that is capable of reducing or preventing at least one symptom, type of injury, or resulting damage, from ischemia-reperfusion injury in an individual when administered one or more times over a suitable time period. Suitable doses of proteins, liposomes, antibodies and other agents, including for various routes of administration, are described in detail above. In one aspect, an effective amount of an agent that inhibits ischemia-reperfusion injury to administer to an individual comprises an amount that is capable of inhibiting at least one symptom or damage caused by ischemia-reperfusion injury without being toxic to the individual.

One of skill in the art will be able to determine that the number of doses of an agent to be administered to an individual is dependent upon the extent of the ischemic event and/or the anticipated or observed physiological damage associated with ischemic-reperfusion injury, as well as the response of an individual patient to the treatment. The clinician will be able to determine the appropriate timing for delivery of the agent in a manner effective to reduce the symptom(s) associated with ischemia-reperfusion injury in the individual. Preferably, the agent is delivered within 48 hours, and more preferably 36 hours, and more preferably 24 hours, and more preferably within 12 hours, and more preferably within 6 hours, 5 hours, 4 hours, 3 hours, 2 hours, or 1 hour, or even minutes after the recognition of an ischemic condition in an individual; after an event that causes ischemia or ischemia-reperfusion injury in an individual or that is predicted to cause ischemia or ischemia-reperfusion injury in an individual, which can include administration prior to the development of any symptoms of ischemia-reperfusion injury in the individual. In one embodiment, the agent is administered concomitantly with (at the same time or within minutes or hours of) conventional therapy for ischemia, such as fluid resuscitation. In one embodiment, the agent is administered as soon as it is recognized (i.e., immediately) by the patient or clinician that the patient may suffer from ischemia-reperfusion injury, is suffering from ischemia-reperfusion injury, or will suffer from ischemia-reperfusion injury. Preferably, such administrations are given until signs of reduction of physiological damage or reduction of the symptoms appear, or until the potential for physiological damage due to ischemia-reperfusion has diminished or is eliminated, and/or as needed until any symptoms are gone or arrested.

According to the present invention, the methods of the present invention are suitable for use in an individual that is a member of the Vertebrate class, Mammalia, including, without limitation, primates, livestock and domestic pets (e.g., a companion animal). Most typically, an individual will be a human individual. The term “individual” can be interchanged with the term “subject” or “patient” and refers to the subject of a method according to the invention. Accordingly, an individual can include a healthy, normal (non-diseased) individual, but is most typically an individual who has or is at risk of developing ischemia-reperfusion injury or a symptom or indicator thereof as described herein.

The following experimental results are provided for purposes of illustration and are not intended to limit the scope of the invention.

### EXAMPLES

### Example 1

The following example demonstrates that liposomes comprising cholesterol, phosphatidylcholine and phosphoglycerol block ischemia-reperfusion injury in vivo in immune competent animals, and that an antibody that selectively binds to annexin-4 readily transfers the capacity of B cell-deficient mice to develop ischemia-reperfusion injury.

Following studies in a model of intestinal ischemia-reperfusion using Crry-Ig to block complement activation after initiation of reperfusion (Rehrig et al., 2001), the present inventors initially set out to determine whether complement receptor CR2, first expressed on B cells during the latter stages of development in the peripheral lymphocyte compartment, might play a role in the generation of the pathogenic natural antibodies that initiate intestinal ischemia-reperfusion injury. The inventors found that Cr2−/− mice did not demonstrate severe intestinal injury that was readily observed in control Cr2+/+ mice following ischemia-reperfusion, despite having identical total serum levels of IgM and total IgG (Fleming et al., 2002). Importantly, pretreatment of Cr2−/− mice prior to the ischemic phase with IgM and IgG purified from the serum of wild type C57BL/6 mice reconstituted all key features of ischemia-reperfusion injury. This result strongly suggested that the defect in Cr2−/− mice involves the failure to develop this subset of pathogenic natural antibodies rather than a failure of CR2 expression on inflammatory cells in the intestine.

Based on these initial studies, the present inventors compared the reactivity of polyclonal antisera from individual and pooled sera from wild type and CR2 deficient mice against freshly isolated and apoptotic intestinal epithelial cells, both by flow cytometry as well as Western blot analysis. The inventors chose to use apoptotic and non-apoptotic intestinal cells as the initial model because they are the relevant target of natural Abs, intestinal epithelial cells under apoptosis during ischemia-reperfusion injury (Ikeda et al., 1998), and these cells can be cultured under conditions that either maintain their cell-cell contacts and viability or, alternatively, induce apoptosis (Grossman et al., 1998; Strater et al., 1996). In parallel, the inventors examined reactivity with a traditional model of apoptosis, which is murine thymocytes, because of the more well-characterized control of apoptosis in these cells (Ashwell et al., 2000).

As a central component of the studies, the inventors prepared novel B cell hybridomas from wild type mice and screened them for reactivity with intestinal epithelial cells by both flow cytometry and Western blot analysis. The experiments described below utilized these novel monoclonal antibodies that were produced by fusing B cells isolated from spleen, peritoneum and mesenteric lymph nodes of wild type mice with the Sp2/0-Ag14 cell line.

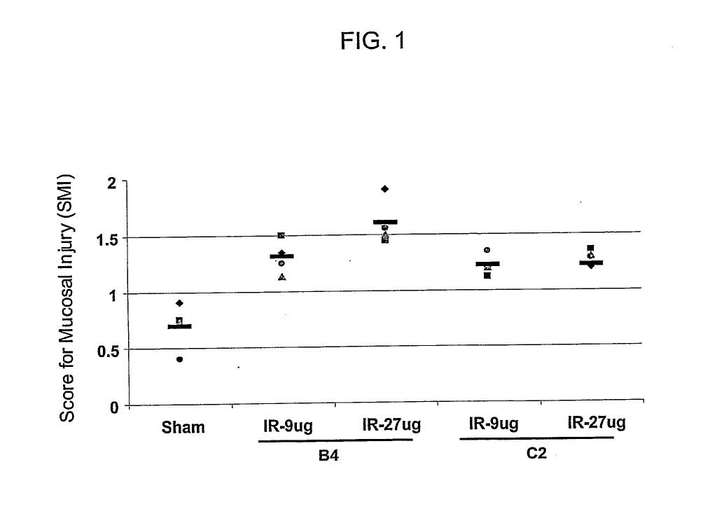

In the first series of experiments, these monoclonal antibodies were administered to Rag−/− mice, which have no immunoglobulin or B cells. Monoclonal antibodies were infused into Rag−/− mice that were then induced to undergo intestinal ischemia-reperfusion injury as described above (see also Example 3). Results of a representative experiment are shown in FIG. 1. Referring to FIG. 1, B4 is an antibody that specifically recognizes annexin-4; C2 is an antibody that recognizes a range of phospholipids.

Several monoclonal antibodies were able to readily transfer the capacity of Rag−/− mice to develop intestinal ischemia-reperfusion injury. These monoclonal antibodies were then studied utilizing protein and lipid purification, protein sequence analysis, mass spectrometry and antigen array techniques in order to identify their antigens. Notably, the inventors found that monoclonal antibodies which efficiently transferred injury in Rag−/− mice recognized either phospholipids (as exemplified by C2 in FIG. 1) or annexin-4 (as exemplified by B4 in FIG. 1).

Therefore, the inventors reasoned that these antibodies were able to recognize their antigenic determinants on cells that were being stressed during ischemia and beginning to undergo the early stages of apoptosis. Indeed, the inventors have shown in vitro that intestinal epithelial cells undergoing stress and early apoptosis react with these pathogenic antibodies (data not shown). Nevertheless, despite the ability of monoclonal antibodies to transfer injury, the inventors were not certain whether this reactivity was relevant to the disease process in wild type mice, or whether an interesting class of antibodies had been identified that was not involved in the actual disease process.

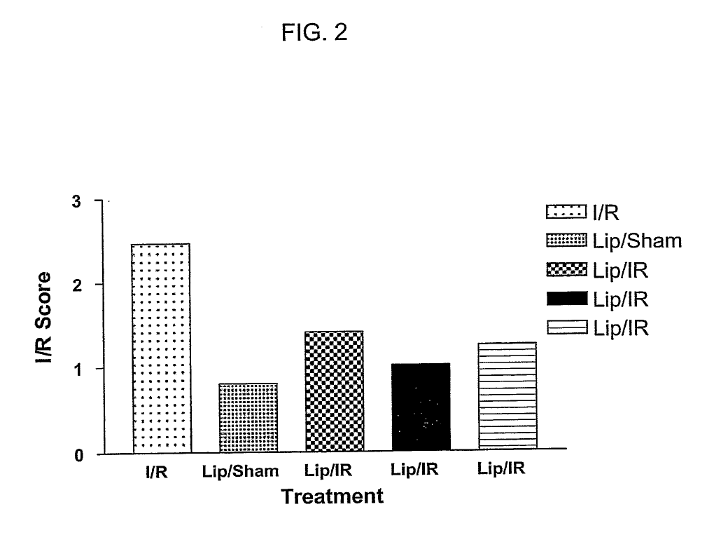

To address this issue, the inventors created compounds that could block the effects of the monoclonal antibodies. This concept was first tested with the antibodies recognizing phospholipids, and liposomes were created composed of cholesterol and the phospholipids phosphatidylcholine and phosphoglycerol. Importantly, when given to wild type mice, either systemically or into the intestinal lumen, the inventors found that the development of intestinal ischemia-reperfusion injury in immune competent mice could be nearly completely blocked (FIG. 2). Thus, of all the potential targets of pathogenic natural antibodies, the epitopes displayed on this type of liposome are essential for reperfusion injury. Mice, and presumably humans, can be shown to demonstrate natural antibody reactivity with phospholipids utilized in this embodiment as well as annexin-4.

With regard to the anti-protein monoclonal antibody that catalyzes ischemia-reperfusion injury and specifically recognizes annexin-4 (e.g., referred to herein as MAb B4), it is of substantial interest that this is a phospholipid binding protein (Kaetzel et al., 2001). In vitro experiments with this monoclonal antibody have indicated that annexin-4 binds to phospholipids displayed on the surface of cells in vitro, thus promoting monoclonal antibody binding. Thus, the inventors reasoned that the similar recognition of this protein on the surface of cells by pathogenic natural antibodies during ischemia-reperfusion or following hemorrhagic hypoperfusion is sufficient to activate complement and induce intestinal injury. This can be tested using methodology similar to what is shown in FIG. 2 with phospholipid-containing liposomes.

### Example 2

The following example describes the optimization of a liposome formulation of the present invention with regard to size, and describes optimization of annexin-4/lipid/phospholipid compositions in order to maximize clinical benefit and systemic delivery capabilities.

In these experiments, additional candidate therapeutic liposomes are developed that contain different ratios and types of phospholipids, with and without annexin-4 bound to the external surface. By performing tissue distribution and initial dose-response and efficacy studies in the intestinal ischemia-reperfusion model under the conditions already shown to be effective in FIG. 2, an optimal formulation is developed that provides benefit at the lowest dose.

To prepare the initially effective liposomes described in Example 1 above, a mixture of a molar ratio of 1:1:2 (25:25:50 as a molar percentage of lipids in the liposome, or calculated based on weight of the lipids, the ratio is 1:1:1) of distearoylphosphatidylcholine (PC; 1,2-Distearoyl-sn-Glycero-3-Phosphocholine):distearoylphosphatidyl glycerol (PG; 1,2-Distearoyl-sn-Glycero-3-[Phospho-rac-(1-glycerol)]):cholesterol dissolved in chloroform was dried under stream of N₂. The remaining lipid film was hydrated with PBS. The suspension was submitted to ten freeze-thaw cycles and extruded twenty times through microporous membrane to obtain liposomes with a diameter <0.1 μm. The resulting combined concentration for PC and PG was 44 μg/ml.

To prepare new liposomes, the ratios of each lipid are varied two-fold over a six-fold range to generate nine additional formulations. Each is tested as described below in six mice. If there are improvements in efficacy demonstrated by formulations, or nearly complete protection is not achieved, new formulations are prepared with additional changes in ratios of lipids and ones with additional phospholipids are included.

To prepare annexin-4 used in Example 1 above, the cDNA sequence was taken from Invitrogen clone ID 4947415 inserted in pCMV-SPORT vector. The coding sequence for annexin IV was inserted into the pET-32 Xa/LIC vector using a (LID) vector kit from Novagen. The vector adds a Trx-Tag, His-Tag, and S-Tag to the N-terminus of the protein. These tags are cleavable with Factor Xa leaving a native version of the N-terminus. The vector can optionally add a non-cleavable His-Tag to the C-terminus of the protein. The inventors have a prepared a construct of A4 that includes the His-Tag on the C-terminus, and one that does not. Annexin IV has been expressed in Novagen BL21 (DE3) and Rosetta 2 (DE3) bacteria. The expressed protein is recognizable by polyclonal rabbit anti-mouse annexin IV antibody (Sigma). The coding sequence for annexin IV was also inserted into a pSecTag2/Hygro B vector from Invitrogen for expression in mammalian cells. Expression for expression in mammalian cells was accomplished in Freestyle 293-F cells (Invitrogen), and this protein was specifically recognized by MAb B4. Nucleic acid sequencing has been performed for each construct and the sequence was confirmed to be without mutation and in-frame.

Liposome-annexin-4 complexes are prepared by the pre-incubation of recombinant annexin-4 with the liposomes described above. Determination of the amount bound is performed by centrifugation of liposomes and measurement of annexin-4 in the liposome pellet to detect a bound versus free proportion. For the experiments below, free annexin-4 is one control.

### Example 3

The following example describes the testing of efficacy of therapeutic liposome formulations by pre-treatment of mouse and rat models of hemorrhage-induced intestinal damage as well as the rat intestinal ischemia-reperfusion models.

In these experiments, the same optimized formulation (see Examples 1 or 2 above) is tested in three rodent models that are relevant to reperfusion injury caused by different mechanisms. By infusing the compound prior to the onset of injury, pathogenic antibodies will be bound and their ability to bind to ischemic tissues will be limited.

Wild type C57BL/6 are utilized for these studies. The ischemia-reperfusion model is performed as outlined briefly. Anesthesia is induced with ketamine (16 mg/kg) and xylazine (8 mg/kg) administered by intramuscular injection. All procedures are performed with the animals breathing spontaneously and body temperature maintained at 37° C. using a water-circulating heating pad. To induce ischemia-reperfusion injury, a midline laparotomy is performed prior to a 30 minute equilibration period. The superior mesenteric artery is identified, isolated and a small non-traumatic vascular clamp (Roboz Surgical Instruments, Rockville, Md.) applied for 30 minutes. After this ischemic phase, the clamp is removed under direct visualization and the intestine allowed to perfuse for 2 hours. In these experiments, at times described below, animals are given identical levels of liposomes, annexin-4, or liposome-annexin-4 complexes, by intravenous injection. Sham animals are subjected to the same surgical intervention except they do not undergo superior mesenteric artery occlusion. To control for the effects of liposome and annexin-4 infusions, these are administered to sham treated mice as well. The laparotomy incisions are then sutured and the animals monitored during the reperfusion period. After euthanasia, the small intestine 10-20 cm distal to the gastroduodenal junction is removed for histologic and immunohistochemical analysis as well as for the measurement of inflammatory mediators as described below.

For histology and immunohistochemistry, immediately after euthansia segments of small intestine specimens are fixed in 10% buffered formalin. For analysis, sections are embedded in paraffin, sectioned transversely in 5 μm sections and stained with Giemsa. Score for Mucosal Injury (SMI) is graded on a six-tiered scale as described previously (Eror et al., 1999). In addition, the villus height of at least 10 villi from the same section is measured using an ocular micrometer.

The ex vivo generation of eicosanoids by small intestine tissue is be determined. Briefly, fresh mid-jejunum sections will be minced, washed and resuspended in 37° C. oxygenated Tyrode's buffer (Sigma, St. Louis, Mo.). After incubating for 20 minutes at 37° C., supernatants are collected and stored at −80° C. until assayed. The concentration of leukotriene B₄ (LTB₄) is determined using an enzyme immunoassay (Cayman Chemical, Ann Arbor, Mich.). The tissue protein content will be determined using the bicinchoninic acid assay (Pierce, Rockford, Ill.) adapted for use with microtiter plates. LTB₄ levels are expressed per mg protein per 20 minutes. Supernatants generated for the eicosanoid assays are also used to determine peroxidase activity by measuring oxidation of 3,3′,5,5′ tetramethylbenzedene (TMB). Briefly, supernatants are incubated with equal volumes of TMB peroxidase substrate (Kirkgaard and Perry, Inc, Gaithersburg, Md.) for 45 minutes. The reaction is stopped by the addition of 0.18 M sulfuric acid, and the OD₄₅₀ is determined. The concentration of total peroxidase is determined using horseradish peroxidase (Sigma) as a standard and plotted as pg myeloperoxidase activity per mg tissue.

Hemorrhagic shock experiments are performed using analytic methods as previously described (Fleming and Tsokos, 2004).

### Example 4

The following example describes the establishment of the time period following the onset of the pathogenic process during which the liposomes remain effective in order to determine the “therapeutic window”.

These experiments determine the time period following the onset of injury during which the therapeutic will provide efficacy. This time period is useful for establishing clinical trial design in humans and for determining the range of clinical conditions that can be treated.

The experiments shown above in Example 1 were performed by infusion immediately prior to the release of the clamp. Previously, the inventors have shown that infusion of the complement inhibitor Crry-Ig is effective at least 30 minutes after release of the clamp, in a therapeutically relevant time frame (Rehrig et al., 2001). Thus, there appears to be a period of time after the onset of injury in which the injection of liposomes, annexin-4 or liposome-annexin-4 complexes will be effective. This therapeutic window is determined experimentally by increasing the time after onset of injury by 15 minute increments until a period of time having no protective effect is demonstrated. Without being bound by theory, the present inventors anticipate that this window will be between 30 and 60 minutes.

### Example 5

The following example demonstrates a role for natural IgM antibodies in causing cerebral injury following ischemic stroke in mice, and show that similar antigen specificities that trigger injury in the intestine (see Kulik et al.), trigger injury in the brain.

Natural IgM antibodies play an important role in injury following ischemia and reperfusion (I/R). Specificity against nonmuscle myosin heavy chain type II A and C has previously been shown to be important for causing injury in mouse models of intestine and hindlimb I/R (M C Carroll, F D Moore et. al.), and data presented elsewhere herein show that antibodies recognizing annexin IV or different phospholipids induce injury following intestine I/R in mice (Kulik et al).

In these experiments, described in detail below, mice were subjected to 60 min middle cerebral artery occlusion followed by 24 h reperfusion. Compared to C57BL/6 wt type controls, Rag1−/− mice had significantly smaller cerebral infarct volumes (8.5%+1-5.4% vs. 26%+/−12.8%) and improved survival 24 h post reperfusion. Treatment of Rag1−/− mice individually with anti-phospholipid mAb C2 or anti-annexin IV mAb B4 (100 μg just prior to reperfusion) restored injury following ischemic stroke (22+/−7% and 28+/−13% infarct vol respectively, and not significantly different). Dose response studies with lower mAb concentrations indicated that C2 mAb is more effective than B4 mAb at causing cerebral injury in Rag−/− mice, an opposite trend to that seen in the model of intestine I/R (see Example 1 and FIG. 1), and possibly a reflection of the high lipid content of the brain. Immunofluorescence microscopy demonstrated IgM and C3 deposition on endothelial surfaces in the penumbria region area and within parenchymal areas of the infarcted brain of wt and Rag1−/− mice treated with mAb. There was no detectable IgM or C3 in sections from untreated Rag1−/− mice. Finally, in antibody screens for pathogenic natural antibodies (see abstract by Kulik et al), a hybridoma (D5) reactive against citrulline-modified protein was also isolated. This antibody specificity enhances tissue injury in experimental murine autoimmune arthritis, but failed to produce any significant injury following ischemic stroke in Rag1−/− mice. These data demonstrate that there are a several pathophysiologically important epitopes that are recognized across multiple tissues by a subset of natural antibodies.

### Materials and Methods

### Middle Cerebral Artery Occlusion (MCAO) and Reperfusion.

Eight week old male C57Bl/6 and C57Bl/6 C3 deficient mice (Jackson labs, Bar Harbor, Me.) were used in experiments. Mice were anesthetized with chloral hydrate (350 mg/kg) and xylazine (4 mg/kg) i.p., and the left common carotid artery of each mouse was exposed through a mid-line incision in the neck. The superior thyroid and occipital arteries were divided and a microsurgical clip placed around the origin of the external carotid artery (ECA). The distal end of the ECA was ligated with 6-0 silk and transected, and 6-0 silk was tied loosely around the ECA stump. The clip was then removed, and the blunted tip of a 4-0 nylon suture was inserted into the ECA stump. The loop of the 6-0 silk was tightened around the stump, and the nylon suture advanced into and through the internal carotid artery until it rested in the anterior cerebral artery. After the nylon suture had been placed for 60 minutes, it was pulled back into the ECA, and the incision closed.

### Reperfusion Antibodies.

Phospholipid mAb C2, anti-annexin IV mAb B4, anti-citrulline-modified protein D5 were kindly gifted by Dr VM Holers. Rag1−/− mice were reconstituted with titered doses of Ab (6.5, 25, 100 μg) just prior to reperfusion.

### Clinical Analysis.

Animals were monitored for 24 hours post reperfusion and clinical assessed for neurological deficit. Behavioral/neurological deficit was scored as follows: 0, normal motor function; 1, flexion of torso and contralateral forelimb when animal is lifted by the tail; 2, circling to the contralateral side when held by tail on flat surface, but normal posture at rest; 3, leaning to the contralateral side at rest; 4, no spontaneous motor activity.

### Analysis.

Following IRI, brains were harvested and either stained with 2% triphenyltetrazolium chloride to determine infarct volume, or subjected to histological assessment of complement and immunoglobulin deposition using immunofluorescence.

### Results

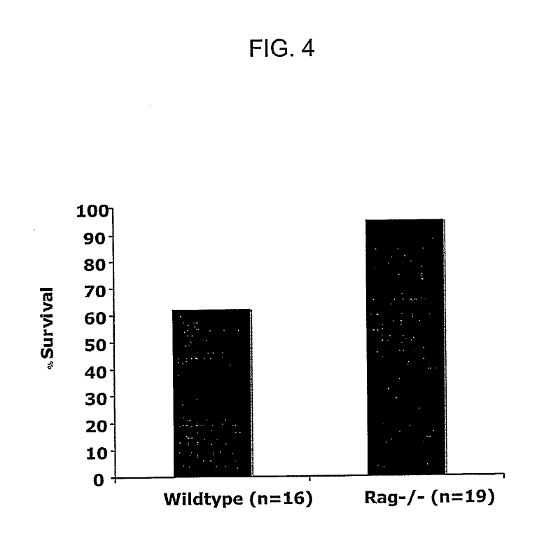

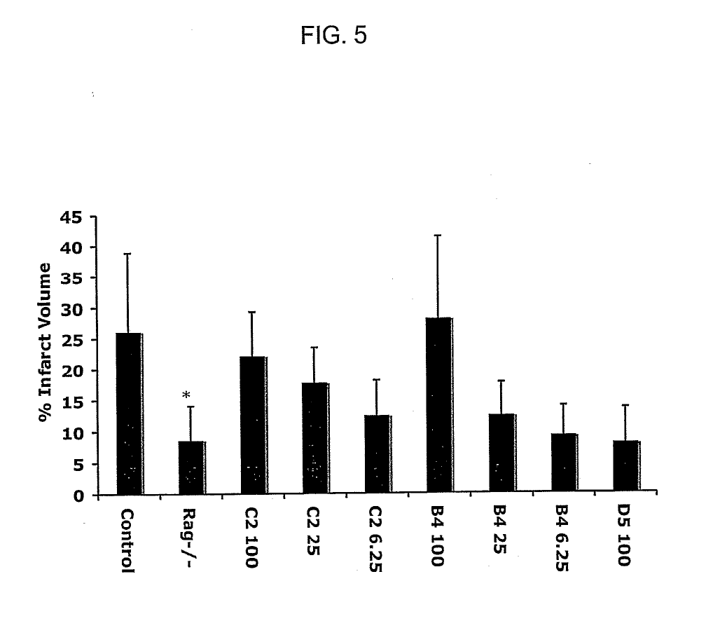

Recent data has shown that animals deficient in immunoglobulin are protected from IRI injury. Given the unique environment of the brain, the inventors sought to assess whether Rag1−/− mice (immunoglobulin deficient) were protected from the injurious effects of ischemic stroke. Referring to FIG. 4, normal C57BL/6 mice and Rag1−/− were subjected to 60 minutes of MCAO induced ischemia and 24 h of reperfusion. All Rag1−/− mice (n=18) survived to the primary end point of 24 h post reperfusion, whereas C57BL/6 mice (n=12) had only a 59% survival rate. The improvement in survival in Rag1−/− compared to control mice was significant (p<0.001). Infarct volume was measured by 2% triphenyltetrazolium chloride (TTC) and the percentage of total cerebral infarct calculated using computerized image analysis (data not shown). Infarct volume in control animals was 26±12.8 compared to 8.5±5.4 in Rag1−/− mice. The inventors have identified three IgM antibodies (referred to herein as C2, B4 and D5), which reconstitute damage in Rag1−/− mice subjected to IRI. In the following experiment, the inventors reconstituted Rag1−/− mice with titred doses of each antibody and investigated whether these antibodies re-constitute damage in a model of ischemic stroke. Infarct volume was again measured by TTC staining and volume calculated using image analysis. Referring to FIG. 5, Rag1−/− significantly protects against cerebral infarct when compared to controls (*p=0.001). Furthermore, reconstitution with C2 and B4, but not D5, induce cerebral infarct in a dose dependant manner. Interestingly, at lower doses, C2 is more effective inducer of damage than B4.

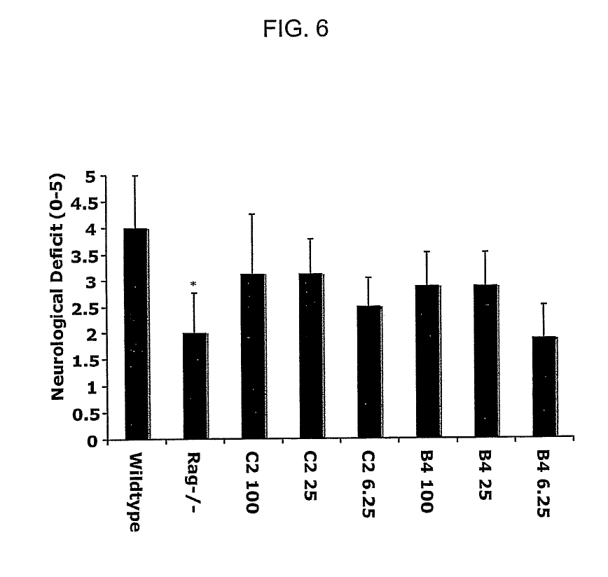

Animals from each group and dose were scored from 0-5 for neurological deficit. Referring to FIG. 6, there was significant reduction in neurological deficit associated with Rag1−/− when compared to wildtype (*p=0.03). As seen in infarct volume observations, animals behaved in a dose dependant manner, with higher doses of both C2 and B4 showing a trend towards a poorer neurological outcome post stroke, although no statistical difference could demonstrated.

Finally, triple confocal immunofluorescence microscopy was used to demonstrate the presence of complement (C3d), IgM and nuclear staining (tro-pro3) within the penumbria area of ipsilateral sections of brains from Rag1−/− and Rag1−/− mice treated with 100 μg C2 mAb (data not shown). Brains were harvested for analysis at 24 hours post ischemic stroke. Complement and IgM deposition could not be demonstrated in untreated Rag1−/− animals at 24 hours post ischemic stroke (data not shown). Rag1−/− animals treated with 100 μg of C2 showed endothelial deposits of both C3d and IgM. Co-localization of complement and IgM showed that both are co-expressed and predominant within the cerebral microvasculature post ischemic stroke. Co-localization of C3d and IgM was seen in the composite image (data not shown).

### Conclusions

In conclusion, in keeping with other IR models, these data show that Rag1−/− mice are protected from damage induced by ischemic stroke as demonstrated by the reduction in infarct volume and improved neurological outcome when compared to wildtype controls. Reconstitution with anti-Annexin IV (MAb B4) and anti-phospholipid (MAb C2) IgM antibodies re-establishes damage post ischemic stoke to levels not significantly different from controls. Furthermore, decreasing titred doses of B4 and C2 result in a reduction in damage, as marked by infarct volume. C2 mAb is more effective than B4 mAb at causing cerebral injury in Rag−/− mice, an opposite trend to that seen in the model of intestine I/R, and possibly a reflection of the high lipid content of the brain. Immunofluorescent analysis shows that reconstitution with IgM Ab's is associated with a concomitant increase in deposition of complement fragments, which are primarily localized to endothelial surfaces. Reconstitution of Rag1−/− mice with D5 (anti-citrulline-modified protein) did not re-establish a damaging phenotype following ischemic stroke. These data, taken together, demonstrate that there are a several pathophysiologically important epitopes that are recognized across multiple tissues by a subset of natural antibodies which induce complement activation and ischemia reperfusion injury.

### Example 6

The following example demonstrates that annexin IV protects against cerebral injury following ischemic stroke in mice.

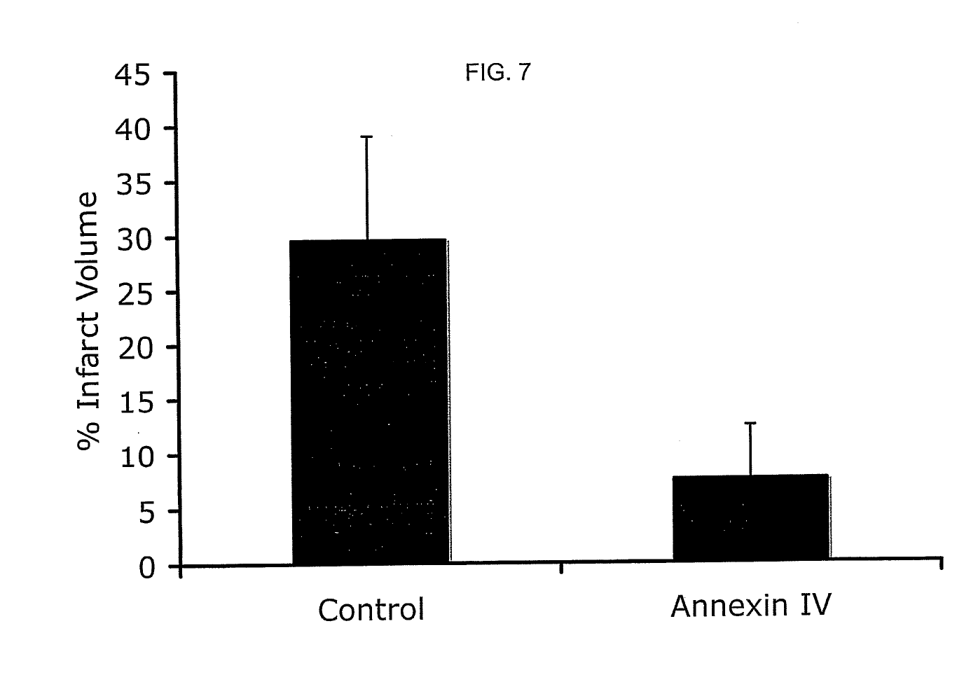

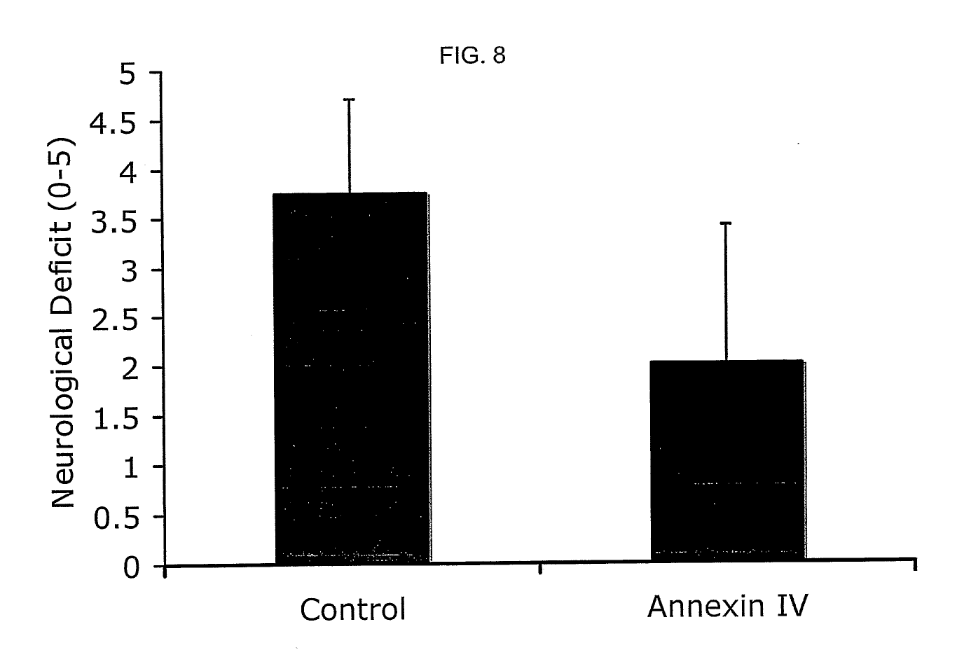

In these experiments, middle cerebral artery occlusion (MCAO) and reperfusion injury were induced in eight week old male C57Bl/6 mice (4 animals per group) as described above in Example 5. Specifically, the mice were subjected to 60 minutes of MCAO-induced ischemia and 24 h of reperfusion. 100 μg of Annexin IV (or PBS) was injected intravenously at 30 min post reperfusion (90 min post start of ischemia). The results are shown in FIGS. 7-8.

Infarct volume was measured by TTC staining and volume calculated using image analysis. Referring to FIG. 7, annexin significantly protects against cerebral infarct when compared to controls.

Animals were monitored for 24 hours post reperfusion and clinical assessed for neurological deficit. Behavioral/neurological deficit was scored as follows: 0, normal motor function; 1, flexion of torso and contralateral forelimb when animal is lifted by the tail; 2, circling to the contralateral side when held by tail on flat surface, but normal posture at rest; 3, leaning to the contralateral side at rest; 4, no spontaneous motor activity. As shown in FIG. 8, mice treated with annexin IV have a significant reduction in neurological deficit when compared to wildtype.

Similar experiments to those described above with annexin IV were also conducted with liposomes composed of cholesterol and the phospholipids phosphatidylcholine and phosphoglycerol, the liposomes being prepared as described in Examples 1 and 2 above. Preliminary results (data not shown) showed that administration of the liposome significantly protected against cerebral infarct when compared to controls not receiving the liposomes (e.g., in one experiment, a mouse had only about 8% infarct compared to controls of about 25-30%).

Without being bound by theory, the present inventors believe that the protection observed as a result of annexin IV and/or lipids would be even better than that described above if administered earlier, such as at the beginning of reperfusion or earlier. Accordingly, it is an embodiment of the invention to administer such therapeutic agents as early as possible after injury is first detected or suspected.

### Example 7

The following example demonstrates that administration of recombinant Annexin IV reduces the level of ischemia reperfusion injury in C57Bl/6 mice. These experiments were conducted in a model of intestinal mesenteric artery ischemia-reperfusion (I/R) injury. Briefly, induction of injury in the model comprises a surgical procedure of opening the abdominal cavity of an anesthetized mouse and occluding the superior mesenteric artery for 30 min, followed by removal of the clamp and 2 hour reperfusion of the tissue. In some of the experiments, 30 min before laparotomy, animals were given 100 μl different doses of B4 or C2 antibody by i.v. injection. In cases where recombinant annexin IV was injected (prepared as in Example 2), it was done right before the reperfusion phase. The liposomes were injected into the lumen 5 min before the reperfusion, and when liposomes were injected i.v. it was done 30 min before reperfusion right after the clamp was inserted. After 2 hours of reperfusion under anesthesia, mice were sacrificed and tissues samples were collected.

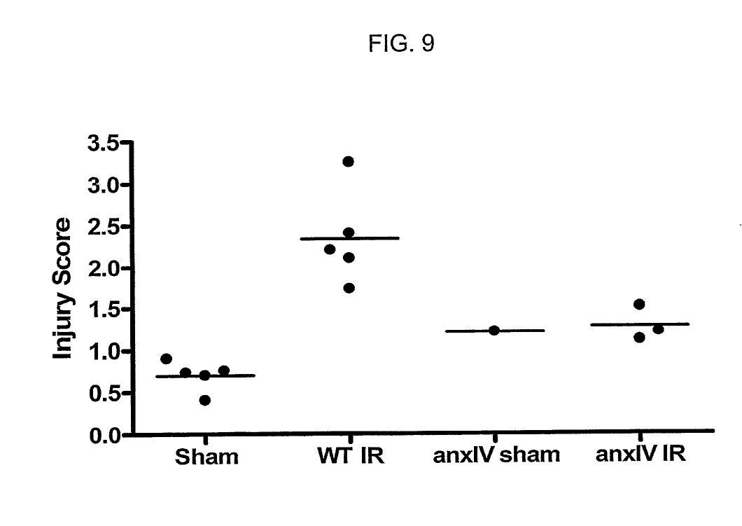

Animals from each group were scored from 0-5 for neurological deficit. Referring to FIG. 9, there was significant reduction in neurological deficit associated with administration of the recombinant annexin IV as compared to control mice and in fact, administration of annexin IV nearly completely protected mice from ischemia-reperfusion injury.

### Example 8

The following example demonstrates that similar antigen specificities that trigger ischemic reperfusion injury in the intestine and the brain enhance damage in rheumatoid arthritis.

In these experiments, passive arthritis was induced by intravenous transfer of a submaximal dose of a cocktail of monoclonal antibodies to CII (Arthrogen-CIA®, Chemicon) and/or the monoclonal antibody B4, which specifically binds annexin-4, as well as purified total IgM from wild type mice. An IgM monoclonal antibody to trinitrophenol-KLH (anti-TNP; BD PharMingen) was administered as a negative control. Arthrogen was titrated to determine the dose that would yield sub-maximal disease in animals for use in combination with test and control antibodies. An intraperitoneal injection of 50 micrograms/mouse of LPS followed three days after administration of each antibody. From days 1 through 14 after the initial transfer, mice were scored daily by an individual blinded to their treatments for signs of arthritis in the paws based on the following scale: 0=no redness or swelling, 1=one digit swollen, 2=two digits swollen, 3=three digits affected, and 4=entire paw swollen with ankylosis. The scores for each of four paws of a mouse were totaled to give a final score with a maximal severity of 16.

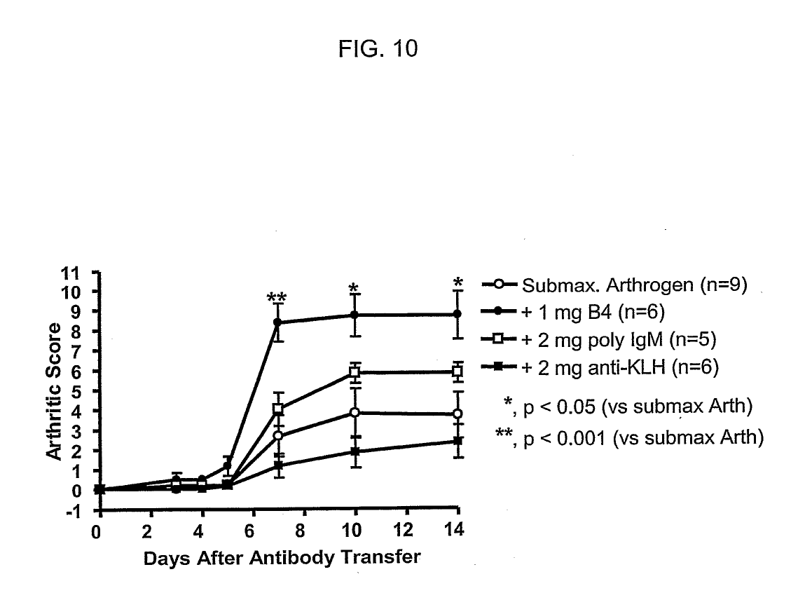

As shown in FIG. 10, administration of monoclonal antibody B4, which binds to annexin IV, results in a significantly greater arthritic score than poly IgM or a submaximal dose of the cocktail of arthritis-inducing monoclonal antibodies, Arthrogen-CIA® (Chemicon International, Inc.; California).

### REFERENCES

1. Fleming et al., 2002, J. Immunol. 169:2126.
2. Fleming and Tsokos, 2004, Curr. Dir. Autoimmunity 7:149.
3. Rehrig et al., 2001, J. Immunol. 167:5921.
4. Ikeda et al., 1998, Gut 42:530.
5. Grossman et al., 1998, Am. J. Pathol. 153:53.
6. Strater et al., 1996, Gastroenterology 110:1776.
7. Ashwell et al., 2000, Ann. Rev. Immunol. 18:309.
8. Kaetzel et al., 2001, Biochemistry 40:4192.
9. Eror et al., 1999, Clin. Immunol. 90:275.
U.S. Provisional Application Ser. No. 60/786,527

Each reference described or cited herein is incorporated herein by reference in its entirety.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1. A method to prevent or treat ischemia-reperfusion injury in an individual, comprising administering to the individual an agent that blocks or inhibits the binding of natural antibodies in the individual to:
a) annexin-4 expressed on the surface of a cell that is in or adjacent to a tissue that is undergoing, or is at risk of undergoing, ischemia-reperfusion injury; and/or
b) a phospholipid expressed on the surface of a cell that is in or adjacent to a tissue that is undergoing, or is at risk of undergoing, ischemia-reperfusion injury.

2. The method of claim 1, wherein the phospholipid is selected from the group consisting of: phosphatidylcholine, phosphoglycerol, lysophosphatidylcholine, phosphatidic acid, phosphatidylethanolamine, phosphatidylserine, and a derivative of any of said phospholipids.

3. (canceled)

4. The method of claim 1, wherein the agent is a liposome or stable lipid moiety comprising said phospholipid.

5. (canceled)

6. The method of claim 1, wherein the agent is a liposome or stable lipid moiety comprising a phospholipid selected from the group consisting of: phosphatidylcholine, phosphoglycerol, lysophosphatidylcholine, phosphatidic acid, phosphatidylethanolamine, phosphatidylserine, and a derivative of any of said phospholipids.

7. (canceled)

8. (canceled)

9. The method of claim 1, wherein the liposome or stable lipid moiety comprises phosphotidylcholine, phosphoglycerol and cholesterol.

10. The method of claim 9, wherein the ratio of phosphotidylcholine:phosphoglycerol:cholesterol is 1:1:2.

11. The method of claim 1, wherein the agent is an isolated annexin-4 protein or biologically active homologue thereof that binds to a phospholipid or comprises at least one conformational epitope bound by a natural antibody in the individual.

12. The method of claim 1, wherein the agent is an isolated annexin-4 protein.

13. The method of claim 1, wherein the agent is an annexin-4-liposome complex or annexin-4-stable lipid moiety complex.

14. The method of claim 13, wherein the liposome or stable lipid moiety portion of the complex comprises a phospholipid selected from the group consisting of phosphatidylcholine, phosphoglycerol, lysophosphatidylcholine, phosphatidic acid, phosphatidylethanolamine, phosphatidylserine, and a derivative of any of said phospholipids.

15. (canceled)

16. The method of claim 13, wherein the liposome or stable lipid moiety comprises phosphotidylcholine, phosphoglycerol and cholesterol.

17. The method of claim 1, wherein the agent is a non-complement-fixing antibody or antigen-binding fragment thereof that selectively binds to annexin-4 and prevents the binding of a natural antibody to the annexin-4.

18. The method of claim 1, wherein the agent is a non-complement-fixing antibody or antigen-binding fragment thereof that blocks or inhibits the binding of a natural antibody to said phospholipid.

19. The method of claim 1, wherein the agent is a non-complement-fixing antibody or antigen-binding fragment thereof that blocks or inhibits the binding of a natural antibody to phosphotidylcholine and/or phosphoglycerol.

20. The method of claim 1, wherein the agent is a drug that competitively inhibits the binding of a natural antibody to said phospholipid.

21. The method of claim 1, wherein the agent is a drug that competitively inhibits the binding of a natural antibody to phosphotidylcholine, phosphoglycerol or annexin-4.

22. The method of claim 1, wherein the agent is a protein or polypeptide that competitively inhibits the binding of a natural antibody to said phospholipid.

23. The method of claim 1, wherein the agent is a protein or polypeptide that competitively inhibits the binding of a natural antibody to phosphotidylcholine, phosphoglycerol or annexin-4.

24. A method to prevent or treat ischemia-reperfusion injury in an individual, comprising administering to the individual a liposome or stable lipid moiety comprising one or more phospholipids.

25. The method of claim 24, wherein the phospholipids are selected from the group consisting of: phosphatidylcholine, phosphoglycerol, lysophosphatidylcholine, phosphatidic acid, phosphatidylethanolamine, phosphatidylserine, and a derivative of any of said phospholipids.

26. (canceled)

27. (canceled)

28. The method of claim 24, wherein the liposome or stable lipid moiety comprises phosphotidylcholine, phosphoglycerol and cholesterol.

29. The method of claim 28, wherein the ratio of phosphotidylcholine:phosphoglycerol:cholesterol is 1:1:2.

30. A method to prevent or treat ischemia-reperfusion injury in an individual, comprising administering to the individual an isolated annexin-4 protein or biologically active homologue thereof that binds to a phospholipid or comprises at least one conformational epitope bound by a natural antibody in the individual.

31. A method to prevent or treat ischemia-reperfusion injury in an individual, comprising administering to the individual an annexin-4-liposome complex, wherein the liposome portion of the complex comprises one or more phospholipids.

32. The method of claim 31, wherein the phospholipid is selected from the group consisting of: phosphatidylcholine, phosphoglycerol, lysophosphatidylcholine, phosphatidic acid, phosphatidylethanolamine, phosphatidylserine, and a derivative of any of said phospholipids.

33. (canceled)

34. The method of claim 24, wherein the ischemia-reperfusion injury is catalyzed by natural antibodies in the individual.

35. The method of claim 1, wherein the ischemia-reperfusion injury is selected from the group consisting of: intestinal ischemia-reperfusion injury, renal ischemia-reperfusion injury, cardiac ischemia-reperfusion injury, ischemia-reperfusion injury of other internal organs such as the lung or liver, central nervous system ischemia-reperfusion injury, ischemia-reperfusion injury of the limbs or digits, or ischemia-reperfusion injury of any transplanted organ or tissue.

36. The method of claim 1, wherein the ischemia-reperfusion injury is associated with an autoimmune disease.

37. The method of claim 1, wherein the ischemia-reperfusion injury is associated with a disease or condition selected from the group consisting of: stroke, traumatic brain injury, spinal cord injury, trauma-induced hypovolemic shock, and rheumatoid arthritis.

38. The method of claim 1, wherein the agent is administered with a pharmaceutically acceptable carrier.

39. The method of claim 1, wherein the agent is administered by a route selected from the group consisting of: nasal, inhaled, intratracheal, topical, and systemic route.

40. A method treat ischemia-reperfusion injury or a disease or condition associated with ischemia-reperfusion injury in an individual, comprising administering to the individual a therapeutic agent for the treatment of ischemia-reperfusion injury or a disease or condition associated with ischemia-reperfusion injury, wherein the agent is linked to a targeting agent that selectively binds to annexin-4 expressed on the surface of a cell that is in or adjacent to a tissue that is undergoing, or is at risk of undergoing, ischemia-reperfusion injury, or to a phospholipid expressed on the surface of a cell that is in or adjacent to a tissue that is undergoing, or is at risk of undergoing, ischemia-reperfusion injury.

41. The method of claim 40, wherein the targeting agent is an antibody or antigen-binding fragment thereof, wherein the antibody is a competitive inhibitor of an antibody that selectively binds to annexin-4 expressed on the surface of a cell or to a phospholipid expressed on the surface of a cell and that catalyzes the initiation and development of ischemia-reperfusion injury.

42. The method of claim 40, wherein the targeting agent is an antigen-binding fragment or non-pathogenic form of an antibody that selectively binds to annexin-4 expressed on the surface of a cell or to a phospholipid expressed on the surface of a cell and that catalyzes the initiation and development of ischemia-reperfusion injury.

43-48. (canceled)

49. An isolated antibody that selectively binds to annexin-4, wherein the antibody induces ischemia-reperfusion injury in an animal.

50. The isolated antibody of claim 49, wherein the antibody is MAb B4.

51. An isolated antibody that selectively binds annexin-4, wherein the antibody competitively inhibits the binding of the antibody of claim 49 to annexin-4, and wherein the antibody does not induce ischemia-reperfusion injury in an animal.

52. An isolated antibody that selectively binds to at least one phospholipid, wherein the antibody induces ischemia-reperfusion injury in an animal.

53. The isolated antibody of claim 52, wherein the antibody is MAb C2.

54. An isolated antibody that selectively binds to at least one phospholipid, wherein the antibody competitively inhibits the binding of the antibody of claim 52 to the phospholipid, and wherein the antibody does not induce ischemia-reperfusion injury in an animal.

55. A liposome or stable lipid moiety that inhibits ischemia-reperfusion injury in an individual, wherein the liposome or stable lipid moiety consists essentially of phosphotidylcholine, phosphoglycerol and cholesterol.

56. The liposome or stable lipid moiety of claim 55, wherein the ratio of phosphotidylcholine:phosphoglycerol:cholesterol is 1:1:2.

57. An annexin-4 liposome complex, wherein the liposome comprises at least one phospholipid.

58. The annexin-4 liposome complex of claim 57, wherein the phospholipid is selected from the group consisting of: phosphatidylcholine, phosphoglycerol, lysophosphatidylcholine, phosphatidic acid, phosphatidylethanolamine, phosphatidylserine, and a derivative of any of said phospholipids.

59. (canceled)

60. (canceled)

61. The annexin-4 liposome complex of claim 57, wherein the liposome comprises phosphotidylcholine, phosphoglycerol and cholesterol.

62. The annexin-4 liposome complex of claim 57, wherein the ratio of phosphotidylcholine:phosphoglycerol:cholesterol is 1:1:2.

63. A method to treat an autoimmune disease, comprising administering to the individual an agent that blocks or inhibits the binding of natural antibodies in the individual to:
a) annexin-4 expressed on the surface of a cell that is in or adjacent to a tissue that is undergoing, or is at risk of undergoing, ischemia-reperfusion injury; and/or
b) a phospholipid expressed on the surface of a cell that is in or adjacent to a tissue that is undergoing, or is at risk of undergoing, ischemia-reperfusion injury.

64. The method of claim 63, wherein the autoimmune disease is rheumatoid arthritis.

65. The method of claim 63, wherein the agent is a liposome or stable lipid moiety comprising said phospholipid.

66. The method of claim 63, wherein the agent is a liposome or stable lipid moiety comprising a phospholipid selected from the group consisting of: phosphatidylcholine, phosphoglycerol, lysophosphatidylcholine, phosphatidic acid, phosphatidylethanolamine, phosphatidylserine, and a derivative of any of said phospholipids.

67. (canceled)

68. The method of claim 63, wherein the agent is an isolated annexin-4 protein or biologically active homologue thereof that binds to a phospholipid or comprises at least one conformational epitope bound by a natural antibody in the individual.

69. The method of claim 63, wherein the agent is an annexin-4-liposome complex or annexin-4-stable lipid moiety complex.
